(19) United States
(12) Patent Application Publication
(10) Pub. No.: US 2010/0008923 A1
(43) Pub. Date: Jan. 14, 2010

(54) Organic Compounds

(75) Inventor: Michael Shultz, Cambridge, MA (US)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: Novartis AG

(21) Appl. No.: 12/305,427

(22) PCT Filed: Jun. 25, 2007
(86) PCT No.: PCT/US07/72004

§ 371 Date: Dec. 18, 2008

Related U.S. Application Data
(60) Provisional application No. 60/816,459, filed on Jun. 26, 2006.

(51) Int. Cl. A61K 31/714; A61K 31/525; A61K 31/51; A61P 35/00; A61K 38/02; A61K 31/4415; A61K 39/395

(52) U.S. Cl. 424/139.1; 514/52; 514/251; 514/276; 514/9

(57) ABSTRACT

The invention relates to pharmaceutical compositions containing inhibitors of histone deacetylase and B vitamins and methods of use thereof, in the treatment of HDAC dependent diseases and for the manufacture of pharmaceutical preparations for the treatment of said diseases.

### FIELD OF USE

The present invention relates to pharmaceutical compositions containing inhibitors of histone deacetylase and B vitamin molecules and methods of use thereof.

### BACKGROUND

Reversible acetylation of histones is a major regulator of gene expression that acts by altering accessibility of transcription factors to DNA. In normal cells, histone deacetylase (“HDAC”) and histone acetyltransferase together control the level of acetylation of histones to regulate active and inactive regions of a chromosome. Acetylation of lysine residues of histone proteins induces conformational changes by destabilizing nucleosomes and allowing transcription factors access to recognition sequences in DNA. Deacetylation of histones by activity of one or more HDACs seals the chromosomal packing, leading to repression of transcription. Inhibition of HDAC results in the accumulation of hyperacetylated histones, which results in a variety of cellular responses.

Inhibitors of HDAC have been studied for their therapeutic effects on cancer cells and in other proliferative diseases. For example, butyric acid and its derivatives, including sodium phenylbutyrate, have been reported to induce apoptosis in vitro in human colon carcinoma, leukemia and retinoblastoma cell lines. Other inhibitors of HDAC that have been widely studied for their anti-proliferative activities are trichostatin A and trapoxin. Trichostatin A is an antifungal and antibiotic and is a reversible inhibitor of mammalian HDAC. Trapoxin is a cyclic tetrapeptide, which is an irreversible inhibitor of mammalian HDAC. Thalidomide has also recently been reported to target HDAC.

Chemotherapeutic agents act on normal growing cells as well as on neoplastic tissue, however, and are toxic to rapidly dividing normal cells as well as to malignant cells. Common immediate side effects are nausea and vomiting, frequently followed by delayed side effects commencing about one month after administration of the therapeutic agent, such as myelosuppression, a condition in which bone marrow activity is decreased resulting in decreased production of blood cells. Such side effects interfere with effective cancer chemotherapy, causing a patient to postpone subsequent rounds of treatment and/or reduce treatment dose. While recent chemotherapeutic agents have reduced side effects compared to older agents, there remains a need to reduce or eliminate side effects of existing agents, so that greater doses and longer protocols or repeated rounds are available to cancer patients.

There remains a need for methods of treating proliferative diseases, including cancerous solid tumors, leukemias, and lymphomas, to ameliorate or reduce undesirable side effects.

### INVENTION DISCLOSURE

The present invention provides in one embodiment a method of treating a subject having a tumor, cell mass or a target cell, the method having the steps of administering to a subject an inhibitor of a histone deacetylase (HDAC) and a B vitamin molecule. A related embodiment further involves after administering to the subject, observing a decrease in proliferation of the tumor, cell mass or target cell compared to a control similarly administered the HDAC inhibitor or the vitamin alone. Observing the decrease in proliferation of the target cell is determined by analyzing inhibition of at least one parameter selected from the group of: tumor size; metastasis; tumor necrosis; cell proliferation rate; and cell apoptosis. In embodiments related to these uses and methods, the subject is a mammal or mammalian cell, for example, the subject is a human.

In embodiments related to these uses and methods, the tumor, cell mass or target cell is present in at least one disease selected from the group of: a proliferative disease, a hyperproliferative disease, a cardiovascular disease, a disease of the immune system, a disease of the central nervous system, a disease of the peripheral nervous system, and a disease associated with misexpression of a gene. In a related embodiment, the cardiovascular disease is heart failure. In a related embodiment, the proliferative disease is a benign or malignant tumor, a carcinoma of the brain, kidney, liver, adrenal gland, bladder, breast, stomach (especially gastric tumors), ovaries, esophagus, colon, rectum, prostate, pancreas, lung, vagina, thyroid, sarcoma, glioblastomas, lymphoma, multiple myeloma or gastrointestinal cancer, colon carcinoma or colorectal adenoma, a tumor of the neck and head, an epidermal hyperproliferation, psoriasis, prostate hyperplasia, a neoplasia, preferably mammary carcinoma, or a leukemia.

In another related embodiment, the hyperproliferative disease is at least one selected from the group of: leukemias, hyperplasias, fibrosis (including pulmonary, and also other types of fibrosis, such as renal fibrosis), angiogenesis, psoriasis, atherosclerosis and smooth muscle proliferation in the blood vessels, such as stenosis or restenosis following angioplasty.

In yet another related embodiment, the immune condition is at least one selected from the group of: rheumatoid arthritis, Crohn's disease, multiple sclerosis, psoriasis, and Type I diabetes. In a further related embodiment, the immune condition is immune rejection of a transplanted allogenic graft of organ or tissue.

In other embodiments related to these uses and methods, the disease to be treated is associated with persistent angiogenesis, such as psoriasis; Kaposi's sarcoma; restenosis, e.g., stent-induced restenosis; endometriosis; Crohn's disease; Hodgkin's disease; leukemia; arthritis, such as rheumatoid arthritis; hemangioma; angiofibroma; eye diseases, such as diabetic retinopathy and neovascular glaucoma; renal diseases, such as glomerulonephritis; diabetic nephropathy; malignant nephrosclerosis; thrombotic microangiopathic syndromes; transplant rejections and glomerulopathy; fibrotic diseases, such as cirrhosis of the liver; mesangial cell-proliferative diseases; arteriosclerosis; injuries of the nerve tissue; and for inhibiting the re-occlusion of vessels after balloon catheter treatment, for use in vascular prosthetics or after inserting mechanical devices for holding vessels open, such as, e.g., stents, as immunosuppressants, as an aid in scar-free wound healing, and for treating age spots and contact dermatitis.

In embodiments related to these uses and methods, the tumor, cell mass or target cell is present in or is associated with an HDAC dependent disease, and the HDAC is at least one selected from the group of HDAC1, HDAC2, HDAC3, HDAC4, HDAC5, HDAC6, HDAC7, HDAC8, HDAC9, HDAC10 and HDAC11. In a related embodiment, the protein HDAC is selected from the group of HDAC1, HDAC2, HDAC6 and HDAC8.

In embodiments related to these uses and methods, the inhibitor of the HDAC includes any compound having a structure that interacts with a histone deacetylase and inhibits HDAC enzymatic activity. Inhibiting HDAC activity is conveniently assayed as inhibiting an identified activity of HDAC, for example, inhibiting removal of an acetyl group from a histone. Alternatively, Inhibiting HDAC activity is assayed as inhibiting deacetylation of other substrates such as tubulin, HSP-90, Hif-1 alpha and p53. In certain embodiments, inhibiting HDAC activity is at least about 50%, at least about 75%, at least about 90%, or at least about 99% compared to activity in the absence of the inhibitor.

In other embodiments related to these uses and methods, the HDAC inhibitor inhibits histone deacetylase at a concentration that is lower than the concentration of the inhibitor that produces another unrelated biological or enzymological effect. In some embodiments the concentration of the HDAC inhibitor used for histone deacetylase inhibitory activity is at least about 2-fold lower, at least about 5-fold lower, at least about 10-fold lower, or at least about 20-fold lower than the concentration that produces an unrelated biological or enzymological effect.

In other embodiments related to these uses and methods, the B vitamin molecule is selected from the group of vitamin B1, vitamin B2, vitamin B3, vitamin B5, vitamin B6, vitamin B9, and vitamin B12. In related embodiments, the B vitamin molecule is selected from the group of vitamin B2, vitamin B3, vitamin B6, vitamin B9, and vitamin B12. In still another related embodiment, the B vitamin molecule is a B vitamin precursor. In yet another related embodiment, the B vitamin molecule is a B vitamin analog or derivative.

In embodiments related to these uses and methods the administering is delivering by a route that is systemic. For example, the route of systemic administration is at least one of: oral, subcutaneous, intramuscular, intraperitoneal, transcutaneous, and intravenous.

In one embodiment of these uses and methods, administering the combination is administering the vitamin and the inhibitor simultaneously. In an alternative embodiment, administering the combination is administering the vitamin and the inhibitor sequentially. In a related embodiment, the doses of the vitamin and the inhibitor are administered at different frequencies. For example: administering the vitamin is more frequent than administering the inhibitor; alternatively, administering the inhibitor is more frequent than administering the vitamin.

In embodiments related to these uses and methods, the dose of the vitamin per subject is at least about 50 micrograms (μg), at least about 80 μg, 90 μg, 100 μg, or at least about 500 μg, at least about 25 milligrams (mg), 30 mg, 40 mg, or at least about 50 mg, to at least about 500 mg.

In an embodiment related to these uses and methods, administering further includes an amount of the HDAC inhibitor/subject/day that is greater and produces fewer side effects than the same amount absent the vitamin.

An embodiment of the invention provides a use of a combination of an HDAC inhibitor and a B vitamin molecule as an anti-cancer treatment. A related embodiment further involves measuring inhibition of at least one parameter selected from the group consisting of: rate of increase in tumor size; rate of increase in tumor number (metastasis); and rate of proliferation of transformed cells.

In certain embodiments, the invention provides a kit for treating a proliferative or a hyperproliferative disorder, the kit including each of an HDAC inhibitor and a B vitamin molecule, and also includes a container. In a related embodiment, each of the HDAC inhibitor and the B vitamin molecule are present in the kit in a unit dose. In another related embodiment, the kit also includes instructions for use. In a related embodiment, the dose is in an orally available tablet. In another related embodiment, the dose is contained in a vial for parenteral administration.

An embodiment of the invention provides a pharmaceutical composition including an HDAC inhibitor and a B vitamin molecule. In a related embodiment, the pharmaceutical composition includes each of the HDAC inhibitor and the B vitamin molecule in an effective dose. In another related embodiment, the pharmaceutical composition further includes a pharmaceutically acceptable buffer. In another related embodiment, the pharmaceutical composition is present in a unit dose.

The compounds of the present invention are suitable as active agents in pharmaceutical compositions that are efficacious particularly for treating cellular proliferative ailments and/or ailments associated with misregulated gene expression. The pharmaceutical composition in various embodiments has a pharmaceutically effective amount of the present active agent along with other pharmaceutically acceptable excipients, carriers, fillers, diluents and the like. The phrase, “pharmaceutically effective amount” as used herein indicates an amount necessary to administer to a host, or to a cell, issue, or organ of a host, to achieve a therapeutic result, especially an anti-tumor effect, e.g., inhibition of proliferation of malignant cancer cells, benign tumor cells or other proliferative cells, or of any other HDAC dependent disease.

### HDAC Inhibitor Compounds

The terms “histone deacetylase inhibitor”, “inhibitor of histone deacetylase”, or “HDAC inhibitor” as used herein refers to any and all compounds having a structure that is capable of a function of interacting with a histone deacetylase and inhibiting its enzymatic activity. “Inhibiting histone deacetylase enzymatic activity” means reducing the ability of a histone deacetylase to remove an acetyl group from a protein, for example, from a histone, or for example, from a tubulin, from HSP-90, from Hif-1 alpha or from p53. Further, reducing histone deacetylase activity is at least by about 50%, at least by about 75%, at least by about 90%, at least by about 95%, or at least by about 99%, compared to histone deacetylase activity in the absence of the inhibitor.

Further, the inhibitor in certain embodiments inhibits histone deacetylase at a concentration that is lower than the concentration of the inhibitor that produces another, unrelated biological or enzymological effect. For example, the concentration of the inhibitor for histone deacetylase inhibitory activity is at least 2-fold lower, at least 5-fold lower, at least 10-fold lower, or at least 20-fold lower than the concentration that produces an unrelated biological or enzymological effect.

Further, as used herein, this term includes without limitation any HDAC inhibitor previously described, such as compounds found in U.S. Pat. Nos. 6,831,061 (Lee et al.); 6,800,638 (Georges et al.); 6,399,568 (Nishino et al.); 6,124,495 (Neiss et al.); and 5,939,455 (Rephaeli).

Accordingly, in one embodiment, HDAC inhibitors are substituted apicidin derivatives represented by the general formula below, as shown in U.S. Pat. No. 6,831,061:

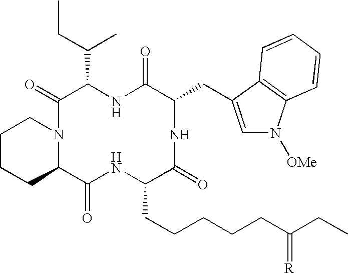

In one embodiment, HDAC inhibitors are tetrahydropyridine derivatives represented by the general formula below, as shown in U.S. Pat. No. 6,800,638:

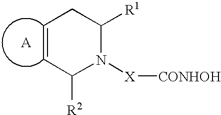

In one embodiment, HDAC inhibitors are cyclic tetrapeptide derivatives represented by the general formula below, as shown in U.S. Pat. No. 6,399,568:

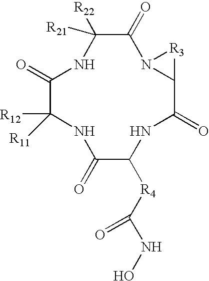

In one embodiment, HDAC inhibitors are unsaturated oxyalkylene esters represented by the general formula below, as shown in U.S. Pat. No. 6,124,495:

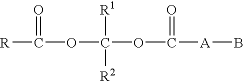

In one embodiment, HDAC inhibitors are oxyalkylene diester butyric acid derivatives represented by the general formulae below, as shown in U.S. Pat. No. 5,939,455:

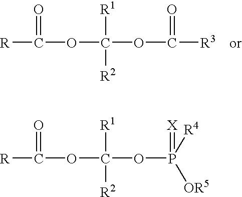

In one embodiment, HDAC inhibitor compounds are hydroxamate derivatives represented by the general formulae below, as shown in PCT publication WO 02/22577:

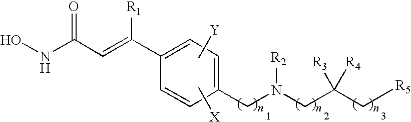

HDAC inhibitors further include compounds such as hydroxamic acids, hydroxamates, hydroxyamides, cyclic peptides, benzamides, benzimidazoles, short-chain fatty acids, mercaptomides, carbamic acids, carbonyls, piperazinyls, piperidinyls, morpholinyls, sulfonyls, amines, amides, valproic acids, oximes, dioxanes, epoxides, lactams, and depudecin.

Examples of HDAC inhibitors that are hydroxamic acids and hydroxamic acid derivatives include, but are not limited to, trichostatin A (TSA), suberoylanlide hydroxamic acid (SAHA), oxamflatin, suberic bishydroxamic acid (SBHA), m-carboxy-cinnamic acid bishydroxamic acid (CBHA), and pyroxamide. Further examples HDAC inhibitors that are hydroxamic acids and hydroxamic acid derivatives are found in application numbers WO03082288 (Watkins et al.), CA2520611 (Miller et al.), WO2005075466 (Bordogna et al.), WO2005053610 (Miller et al.), US2005124679 (Kim et al.), and WO2005014588 (Dyke et al.).

Examples of HDAC inhibitors that are hydroxamates and hydroxamate derivatives include, but are not limited to, those found in application numbers US2006058553 (Leahy et al.), WO2005097770 (Setti), WO2005058803 (LeBlond et al.), and WO2005040161 (Stunkel et al.). Examples of HDAC inhibitors that are hydroxyamides and hydroxyamide derivatives include, but are not limited to, those found in application numbers WO2006025683 (Lee et al.) and WO2006016680 (Ishibashi et al.). Examples of HDAC inhibitors that are benzimidazoles and benzimidazole derivatives include, but are not limited to, those found in application number WO2004072047 (Urano et al.). Examples of HDAC inhibitors that are mercaptomides and mercaptomide derivatives include, but are not limited to, those found in application numbers WO2006028972 (Ahmed et al.) and WO2005075446 (Koyama et al.). Examples of HDAC inhibitors that are carbamic acids and carbamic acid derivatives include, but are not limited to, those found in application numbers US2006058282 (Finn et al.) and US2005143385 (Watkins et al.). Examples of HDAC inhibitors that are carbonyls and carbonyl derivatives include, but are not limited to, those found in application numbers EP1635800 (Wash et al.), US2005148613 (Van Emelen et al.), WO03099760 (Lan-Hargest et al.), and WO03099789 (Lan-Hargest et al.). Examples of HDAC inhibitors that are piperazinyls, piperidinyls, and morpholinyls and piperazinyl, piperidinyl, and morpholinyl derivatives include, but are not limited to, those found in application numbers ZA200407237 (Van Emelen et al.) and WO2006010749 (Van Brandt et al.). Examples of HDAC inhibitors that are sulfonyls and sulfonyl derivatives include, but are not limited to, those found in application numbers WO03076401 (Van Emelen et al.), US2006030543 (Malecha et al.), and WO2005040101 (Lim et al.). Examples of HDAC inhibitors that are amines and amine derivatives include, but are not limited to, those found in application numbers WO2006010750 (Verdonck et al.), US2005119250 (Angibaud et al.), US2004157841 (Fertig et al.), and US2004162317 (Fertig et al.). Examples of HDAC inhibitors that are amides and amide derivatives include, but are not limited to, those found in application numbers WO2006005955 (Chakravarty et al.), WO2006005941 (Chakravarty et al.), WO2005065681 (Bressi et al.), and WO03070691 (Uesato et al.). Examples of HDAC inhibitors that are valproic acids and valproic acid derivatives include, but are not limited to, those found in application number US2005038113 (Groner et al.). Examples of HDAC inhibitors that are oximes and oxime derivatives include, but are not limited to, those found in application number CA2519301 (Fertig et al.). Examples of HDAC inhibitors that are dioxanes and dioxane derivatives include, but are not limited to, those found in application number WO02089782 (Schreiber et al.). Examples of HDAC inhibitors that are epoxides and epoxide derivatives include, but are not limited to, those found in application numbers US2005282890 (Zheng) and WO03099272 (Lan-Hargest et al.). Examples of HDAC inhibitors that are lactams and lactam derivatives include, but are not limited to, those found in application number US2004077698 (Georges et al.).

Examples of HDAC inhibitors that are cyclic peptides include, but are not limited to, trapoxin A, apicidin and FR901228. Further examples of HDAC inhibitors that are cyclic peptides and cyclic peptide derivatives are found in application numbers US2002120099 (Basting), U.S. Pat. No. 6,656,905 (Mori et al.), and U.S. Pat. No. 6,399,568 (Nishino et al.).

Examples of HDAC inhibitors that are benzamides include but are not limited to MS-27-275 (N-(2-aminophenyl)-4-[N-(pyridin-3-ylmethoxycarbonyl)aminomethyl]benzamide). Further examples of HDAC inhibitors that are benzamides and benzamide derivatives are found in application numbers HK1079042, US2005171103 (Stokes et al.), and HK1046277 (Ishibashi et al.).

Examples of HDAC inhibitors that are short-chain fatty acids include but are not limited to butyrates (e.g., butyric acid, arginine butyrate and phenylbutyrate). Newmark et al. (1994) Cancer Lett. 78:1-5; and Carducci et al. (1997) Anticancer Res. 17:3972-3973. Further examples of HDAC inhibitors that are short-chain fatty acids and short chain fatty-acid derivatives are found in application numbers US2006069157 (Ferrante), WO2005055928 (Chen et al.), and WO9800127 (Rephaeli et al.).

In addition, depudecin which has been shown to inhibit HDAC at micromolar concentrations (Kwon et al. (1998) Proc. Natl. Acad. Sci. USA. 95:3356-3361) also falls within the scope of histone deacetylase inhibitor of the present invention.

In general, HDAC inhibitors are soluble in alcohols such as methanol or ethanol, or in organic solvents such as dimethyl sulfoxide (DMSO). Alternatively, HDAC inhibitors can be complexed with a cyclodextrin, for example 2-hydroxypropyl-β-cyclodextrin, see Hockly et al., Proc Natl Acad Sci USA. 2003; 100(4): 2041-2046, so that the HDAC inhibitor is soluble as the complex in aqueous solutions.

### Use in HDAC Dependent Diseases

The methods of the present invention include compounds that have valuable pharmacological properties and that are useful in the treatment of diseases. In certain embodiments of these uses and methods, these compounds are useful in the treatment of HDAC dependent diseases, e.g., as drugs to treat proliferative diseases.

The phrase “treatment of HDAC dependent diseases” refers to the prophylactic or therapeutic (including palliative and/or curing) treatment of these diseases, including for example, the diseases mentioned below.

The term “use” includes any one or more of the following embodiments of the invention, respectively: the use in the treatment of HDAC dependent diseases; the use for the manufacture of pharmaceutical compositions for use in the treatment of these diseases, e.g., in the manufacture of a medicament; methods of use of derivatives in the treatment of these diseases; pharmaceutical preparations having derivatives for the treatment of these diseases; and derivatives for use in the treatment of these diseases; as appropriate and expedient, if not stated otherwise. In particular, diseases to be treated and are thus preferred for use of a compound of the present invention are selected from HDAC dependent (“dependent” meaning also “supported”, not only “solely dependent”) diseases, including those corresponding proliferative diseases, and those diseases that depend on HDAC1, HDAC2, HDAC3, HDAC4, HDAC5, HDAC6, HDAC7, HDAC8, HDAC9, HDAC10, HDAC11, or an RDAC complex (hereinafter “HDACs”) can therefore be used in the treatment of UDAC dependent diseases. The term “use” further includes embodiments of compositions herein which bind to an RDAC protein sufficiently to serve as tracers or labels, so that when coupled to a fluoro or tag, or made radioactive, can be used as a research reagent or as a diagnostic or an imaging agent.

In certain embodiments, the methods of the present invention are used for treating “HDAC-dependent diseases”, i.e., a disease dependant upon an activity of at least one of the HDACs as described herein. It is envisioned that a use can be a treatment of inhibiting one or a subset of the group HDAC1, HDAC2, HDAC3, HDAC4, HDAC5, HDAC6, HDAC7, HDAC8, HDAC9, HDAC10, and HDAC11, and does not imply that all of these enzymes are inhibited to an equal extent by any of the compounds herein.

Also envisioned herein are demonstrations of the antitumor activity of compounds of the present methods in vivo.

Various embodiments of the compounds of the present methods have valuable pharmacological properties and are useful in the treatment of protein HDAC dependent diseases, e.g., as drugs to treat proliferative and hyperproliferative diseases, and other HDAC dependent diseases as listed throughout this disclosure. Various additional embodiments of the compounds of the present invention have valuable binding properties and are useful in diagnostic and labeling capacities and as imaging agents.

### Assays

The inhibition of HDAC activity may be measured as follows: The baculovirus donor vector pFB-GSTX3 is used to generate a recombinant baculovirus that expresses the HDAC polypeptide. Transfer vectors containing the HDAC coding region are transfected into the DH10Bac cell line (GIBCO) and plated on selective agar plates. Colonies without insertion of the fusion sequence into the viral genome (carried by the bacteria) are blue. Isolated, white colonies are picked and viral DNA (bacmid) is isolated from each of the bacterial clones by standard plasmid purification procedures. Sf9 cells or Sf21 (American Type Culture Collection) cells are then transfected in 25 cm² flasks with the viral DNA using Cellfectin reagent.

Determination of small scale protein expression in Sf9 cells: Virus-containing media is collected from the transfected cell culture and used for infection to increase its titer. Virus-containing media obtained after two rounds of infection is used for large-scale protein expression. For large-scale protein expression 100 cm² round tissue culture plates are seeded with 5×10⁷ cells/plate and infected with 1 mL of virus-containing media (at an approximately MOI of 5). After 3 days, the cells are scraped off the plate and centrifuged at 500 rpm for 5 minutes. Cell pellets from 10-20, 100 cm² plates, are re-suspended in 50 mL of ice-cold lysis buffer (25 mM tris-HCl, pH 7.5, 2 mM EDTA, 1% NP-40, 1 mM DTT, 1 mM P MSF). The cells are stirred on ice for 15 minutes and then centrifuged at 5,000 rpms for 20 minutes.

Purification of GST-tagged proteins: The centrifuged cell lysate is loaded onto a 2 mL glutathione-sepharose column (Pharmacia) and is washed 3× with 10 mL of 25 mM tris-HCl, pH 7.5, 2 mM EDTA, 1 mM DTT, 200 mM NaCl. The GST-tagged proteins are then eluted by 10 applications (1 mL each) of 25 mM tris-HCl, pH 7.5, 10 mM reduced-glutathione, 100 mM NaCl, 1 mM DTT, 10% glycerol and stored at −70° C.

Measure of enzyme activity: HDAC assays with purified GST-HDAC protein are carried out in a final volume of 30 μL containing 15 ng of GST-H4DAC protein, 20 mM tris-HCl, pH 7.5, 1 mM MnCl2, 10 mM MgCl2, 1 mM DTT, 3 μg/mL poly(Glu,Tyr) 4:1, 1% DMSO, 2.0 μM ATP (γ-[³³P]-ATP 0.1 μCi). The activity is assayed in the presence or absence of inhibitors. The assay is carried out in 96-well plates at ambient temperature for 15 minutes under conditions described below and terminated by the addition of 20 μL of 125 mM EDTA. Subsequently, 40 μL of the reaction mixture are transferred onto IMMOBILON-PVDF membrane (Millipore) previously soaked for 5 minutes with methanol, rinsed with water, then soaked for 5 minutes with 0.5% H₃PO₄ and mounted on vacuum manifold with disconnected vacuum source. After spotting all samples, a vacuum is connected and each well-rinsed with 200 μL 0.5% H₃PO₄. Membranes are removed and washed 4× on a shaker with 1.0% H₃PO₄, once with ethanol. Membranes are counted after drying at ambient temperature, mounting in Packard TopCount 96-well frame, and addition of 10 μL/well of MICROSCINT™ (Packard). IC₅₀ values are calculated by linear regression analysis of the percentage inhibition of each compound in duplicate, at 4 concentrations (usually 0.01, 0.1, 1 and 10 μM).

### IC₅₀ Calculations

| | |
|---|---|
| Input: | 3 × 4 μL stopped assay on |
| | IMMOBILON membrane, not |
| | washed |
| background (3 wells): | assay with H₂O instead of enzyme |
| positive control (4 wells): | 3% DMSO instead of compound |
| bath control (1 well): | no reaction mix |

IC₅₀ values are calculated by logarithmic regression analysis of the percentage inhibition of each compound at 4 concentrations (usually 3- or 10-fold dilution series starting at 10 μM). In each experiment, the actual inhibition by reference compound is used for normalization of IC₅₀ values to the basis of an average value of the reference inhibitor:

Normalized IC₅₀=measured IC₅₀ average ref. IC₅₀/measured ref. IC₅₀
Example: Reference inhibitor in experiment 0.4 μM, average 0.3 μM Test compound in experiment 1.0 μM, normalization: 0.3/0.4=0.75 μM

For example, known HDAC inhibitors or a synthetic derivative thereof may be used as reference compounds.

Using this protocol, the compounds of the invention are found to show IC₅₀ values for HDAC inhibition in the range from 0.005-100 μM, or 0.002-50 μM, including, for example, the range from 0.001-2 μM or less.

### Proliferative Diseases

As discussed above, the methods of the present invention are useful for treating proliferative diseases. A proliferative disease includes, for example, a tumor disease (or cancer) and/or any metastases) or a proliferative disease of a blood cell, such as a leukemia or a lymphoma. The inventive methods are useful for treating a tumor which is, for example, a breast cancer, genitourinary cancer, lung cancer, gastrointestinal cancer, esophageal cancer, epidermoid cancer, melanoma, ovarian cancer, pancreas cancer, neuroblastoma, head and/or neck cancer or bladder cancer, or in a broader sense renal, brain or gastric cancer, or a leukemia, or a lymphoma; including (i) a leukemia such as a myelogenous leukemia or an acute leukemia or a chronic leukemia; a lymphoma such as Hodgkin's lymphoma or non-Hodgkin's lymphoma; a breast tumor; an epidermoid tumor, such as an epidermoid head and/or neck tumor or a mouth tumor; a lung tumor, for example a small cell or non-small cell lung tumor; a gastrointestinal tumor, for example, a colorectal tumor; or a genitourinary tumor, for example, a prostate tumor (including a hormone-refractory prostate tumor); or (ii) a proliferative disease that is refractory to the treatment with other chemotherapeutics; or (iii) a tumor that is refractory to treatment with other chemotherapeutics due to multidrug resistance.

TABLE 1
HDAC 1-11 genes with O.M.I.M accession number and
chromosomal locus

| | OMIM | |
|---|---|---|
| Histone deacetylase | accession number | Chromosomal locus |
| HDAC1 | *601241 | 1p34.1 |
| HDAC2 | *605164 | 6q21 |
| HDAC3 | *605166 | 5q31 |
| HDAC4 | *605314 | 2q37.2 |
| HDAC5 | *605315 | Chr.17 |
| HDAC6 | *300272 | Xp11.23 |
| HDAC7A | *606542 | Chr.12 |
| HDAC8 | *300629 | Xq13 |
| HDAC9 | *606543 | 7p21-p15 |
| HDAC10 | *608544 | 22q13.31-q13.33 |
| HDAC11 | *607226 | 3p25.2 |

An HDAC dependent disease is any pathology related to expression of one or more of the genes encoding one of the HDAC proteins or HDAC-associated proteins, or an activity of such as protein, in that inhibition of the protein results in remediation of the pathology. The HDAC genes and proteins are as described in the Online Mendelian Inheritance in Man (O.M.I.M). Inhibition of an HDAC protein provides remediation of an HDAC dependent disease. Table 1 lists the HDAC proteins and the locus of each on the human genome. Table 2 shows HDAC 1-11 GenBank accession numbers for representative amino acid sequences in at least three organismal species when available.

TABLE 2
GenBank accession numbers for exemplary amino acid sequences of
HDAC1-11 proteins

| Histone deacetylase | GenBank amino acid sequence | |
|---|---|---|
| protein | accession number | Source |
| HDAC1 | O60341 | Human |
| | NP_033214 | Mouse |
| | NP_571138 | Zebra fish |
| HDAC2 | NP_032255 | Human |
| | P70288 | Mouse |
| HDAC3 | NP_006302 | Human |
| | NP_034541 | Mouse |
| | NP_957284 | Zebra fish |
| HDAC4 | NP_005648 | Human |
| | NP_989644 | Chicken |
| | AAX52490 | Fruit fly |
| HDAC5 | NP_001015033 | Human |
| | AAS77826 | Porcine |
| | NP_034542 | Mouse |
| HDAC6 | Q9C2B2 | Human |
| | NP_034543 | Mouse |
| | AAH43813 | African clawed |
| | | frog |
| HDAC7 | NP_057680 | Human |
| | AAK11188 | Norway rat |
| | Q8C2B3 | Mouse |
| HDAC8 | Q9BY41 | Human |
| | Q8VH37 | Mouse |
| | AAH55541 | Zebra fish |
| HDAC9 | Q9UKV0 | Human |
| | NP_07738 | Mouse |
| | NP_957110 | Zebra fish |
| HDAC10 | Q969S8 | Human |
| | Q569C4 | Norway rat |
| | NP_954668 | Mouse |
| HDAC11 | Q96DB2 | Human |
| | Q91WA3 | Mouse |

In certain embodiments, the proliferative disease may furthermore be a hyperproliferative condition such as a leukemia, hyperplasia, fibrosis (including pulmonary, and also other types of fibrosis, such as renal fibrosis), angiogenesis, psoriasis, atherosclerosis and smooth muscle proliferation in the blood vessels, such as stenosis or restenosis following angioplasty.

Where a tumor, a tumor disease, a carcinoma or a cancer are mentioned, also metastasis in the original organ or tissue and/or in any other location are implied alternatively or in addition, whatever the location of the tumor and/or metastasis.

The compounds described in the methods herein are selectively toxic or more toxic to rapidly proliferating cells than to normal cells, including, for example, human cancer cells, e.g., cancerous tumors. The compounds have significant antiproliferative effects and promote differentiation, e.g., cell cycle arrest and apoptosis. In addition, the compounds of the methods herein induce p21, cyclin-CDK interacting protein, which induces either apoptosis or G1 arrest in a variety of cell lines.

The following examples are intended to illustrate the invention and are not to be construed as being limitations thereto.

In the following embodiments, general expression can be replaced by the corresponding more specific definitions provided above and below.

In certain embodiments of the pharmaceutical compositions, uses, and methods herein, the use of compounds of the present invention, tautomers thereof or pharmaceutically acceptable salts thereof, the HDAC dependent disease to be treated is a proliferative disease depending on any one or more of the following HDACs, including, for example, HDAC1, HDAC2, HDAC6 and HDAC8.

In other embodiments, the HDAC dependant disease may be a proliferative disease including a hyperproliferative condition, such as leukemias, hyperplasias, fibrosis (including pulmonary, but also other types of fibrosis, such as renal fibrosis), angiogenesis, psoriasis, atherosclerosis and smooth muscle proliferation in the blood vessels, such as stenosis or restenosis following angioplasty.

In other embodiments, the invention provides pharmaceutical compositions, uses, and methods of treating an HDAC dependent disease comprising administering an HDAC inhibitor and a B vitamin molecule, where the disease to be treated is a proliferative disease, including, for example, a leukemia such as a myelogenous leukemia or an acute leukemia or a chronic leukemia, a lymphoma such as Hodgkin's lymphoma or non-Hodgkin's lymphoma, a benign or malignant tumor, a carcinoma of the brain, kidney, liver, adrenal gland, bladder, breast, stomach (including gastric tumors), esophagus, ovaries, colon, rectum, prostate, pancreas, lung (including SCLC), vagina, thyroid, sarcoma, glioblastomas, multiple myeloma or gastrointestinal cancer, especially colon carcinoma or colorectal adenoma, or a tumor of the neck and head, an epidermal hyperproliferation, including psoriasis, prostate hyperplasia, a neoplasia, including those of epithelial character, including mammary carcinoma, or a proliferative disease of a blood cell such as a lymphoma or a leukemia. Also included is a method for the treatment of atherosclerosis, thrombosis, psoriasis, scleroderma and fibrosis.

The use of the methods brings about regression of tumors and prevention or reduction of the formation of tumor metastases (including micrometastases) and the growth of metastases (including micrometastases). In addition these methods are used to treat epidermal hyperproliferation (e.g., psoriasis), in prostate hyperplasia, and to treat neoplasias, including that of epithelial character, for example mammary carcinoma. It is also possible to use the methods of the present invention to treat diseases of the immune system insofar as one or more individual HDAC protein species or associated proteins are involved. Furthermore, the methods of the present invention can be used also to treat diseases of the central or peripheral nervous system where signal transmission by at least one HDAC protein is involved.

The pharmaceutical compositions, uses, and methods of the present invention are also appropriate for therapy of diseases related to transcriptional regulation of proteins involved in signal transduction, such as VEGF receptor tyrosine kinase overexpression. Among these diseases are retinopathies, age-related macula degeneration, psoriasis, haemangioblastoma, haemangioma, arteriosclerosis, muscle wasting conditions such as muscular dystrophies, cachexia, Huntington's syndrome, inflammatory diseases such as rheumatoid or rheumatic inflammatory diseases, including arthritis and arthritic conditions, such as osteoarthritis and rheumatoid arthritis, or other chronic inflammatory disorders such as chronic asthma, arterial or post-transplantational atherosclerosis, endometriosis, and especially neoplastic diseases, for example so-called solid tumors (including cancers of the gastrointestinal tract, the pancreas, breast, stomach, cervix, bladder, kidney, prostate, esophagus, ovaries, endometrium, lung, brain, melanoma, Kaposi's sarcoma, squamous cell carcinoma of head and neck, malignant pleural mesotherioma, lymphoma or multiple myeloma) and liquid tumors (e.g., leukemias).

HDAC proteins share a set of nine consensus sequences. HDAC proteins are classified into two classes based on amino acid sequence: class I proteins such as HDAC1, HDAC2 and HDAC3 have substantial homology to yeast Rpd3; class II proteins such as HDAC4 and HDAC6 show homology to yeast Hda1. A variety of findings facts indicate an association of these proteins with HDAC dependent diseases.

HDAC1 is a protein having 482 amino acids, and is highly conserved in nature, having 60% identity to a yeast transcription factor. It is found at various levels in all tissues, and is involved in transcriptional regulation and cell cycle progression, particularly G1 checkpoint control. HDAC1 interacts physically with and cooperates with RB1, the retinoblastoma tumor suppressor protein that inhibits cell proliferation, and with nuclear transcription factor NFκB.

HDAC2 is also known as YY1-associated factor (YAF1), as it associates with mammalian zinc finger transcription factor YY1. The locus that encodes this protein on the human genome is 6q21, a region of the genome implicated in childhood acute lymphocytic leukemia (ALL) and ulnar ray limb defect. Further, HDAC2 interacts with and is physically associated with BRCA1 in a complex that includes also HDAC1. The common core of this complex functions to repress genes to a silent condition. A different complex is formed during S phase, and histone is deacetylated into heterochromatin following replication.

HDAC3 is known to be expressed in all human tissues and tumor cell lines. Transfection of a human myeloid leukemia line resulted in accumulation of cells at the G2/M boundary phase with aberrant nuclear morphology and increased cell size. The catalytic domain of HDAC3 interacts with the catalytic domain of HDAC4.

HDAC4 deacetylase activity acts on all four core histone proteins, is expressed in prehypertrophic chondrocytes, and regulates chondrocyte hypertrophy, endochondral bone formation and skeletogenesis. HDAC4-null mice display premature ossification. With MIR and CABIN1, HDAC4 constitutes a family of calcium-sensitive transcriptions repressors of MEF-2 (myocyte enhancer factor-2).

HDAC5 is expressed in all tissues tested, with lower expression in spleen and pancreas. The 1,123 amino acid sequence of HDAC5 is 51% identical to HDAC4. Five of 29 colon cancer patients tested serologically positive for antibody to HDAC5. MEF-2 protein interacts with HDAC4 and HDAC5.

HDAC6 is a tubulin deacetylase and is localized exclusively in cytoplasm. This enzyme has potent deacetylase activity for assembled microtubules, and therapeutic intervention into its expression or activity can be associated with a variety of conditions affecting muscle integrity and muscle wasting, such as Huntington's disease and cachexia.

HDAC7A transcript is found predominantly in heart and lung tissues, and to a lesser extent in skeleton muscle. The protein co-localizes with HDAC5 in subnuclear regions.

HDAC8 is a 377 amino acid protein which while possessing the typical nine conserved HDAC blocks of consensus sequence, has sequences at each of the amino and carboxy termini that are distinct from those of other HDAC proteins. It is expressed most strongly in brain. Knockdown of expression by RNAi inhibits growth of human lung, colon, and cervical cancer cell lines. The map position of the encoding gene at Xq13 is located near XIST which is involved in initiation of X chromosome inactivation, and near breakpoints associated with preleukemia conditions. Further, therapeutic intervention into its expression or activity can be associated with a variety of conditions affecting inflammatory diseases such as various arthritic conditions, e.g., rheumatoid arthritis.

HDAC9 is known also as 7B, MITR, and KIAA0744. It is expressed most actively in brain, and to a lesser extent in heart and smooth muscle, and very little in other tissues. This protein interacts with HDAC1 and is a repressor of transcription. A longer isoform contains 1,011 amino acids and a shorter form, known as 9a, contains 879 amino acids, lacking 132 residues at the C-terminus, predominates in lung, liver and skeletal muscle.

HDAC10 is found in two splice variants of 669 and 649 amino acids. The protein represses transcription from a thymidine kinase promoter and interacts with HDAC3.

HDAC11 is a 347 amino acid protein that is expressed most highly in brain, heart, skeletal muscle, kidney and testis. It partitions with nuclear extracts.

The methods of the present invention can also be used to prevent or treat diseases that are triggered by persistent angiogenesis, such as psoriasis; Kaposi's sarcoma; restenosis, e.g., stent-induced restenosis; endometriosis; Crohn's disease; Hodgkin's disease; leukemia; arthritis, such as rheumatoid arthritis; hemangioma; angiofibroma; eye diseases, such as diabetic retinopathy and neovascular glaucoma; renal diseases, such as glomerulonephritis; diabetic nephropathy; malignant nephrosclerosis; thrombotic microangiopathic syndromes; transplant rejections and glomerulopathy; fibrotic diseases, such as cirrhosis of the liver; mesangial cell-proliferative diseases; arteriosclerosis; injuries of the nerve tissue; and for inhibiting the re-occlusion of vessels after balloon catheter treatment, for use in vascular prosthetics or after inserting mechanical devices for holding vessels open, such as, e.g., stents, as immunosuppressants, as an aid in scar-free wound healing, and for treating age spots and contact dermatitis.

### B Vitamin Molecules

A “B vitamin molecule”, as used herein, refers to any or all of a complex of several vitamins that were discovered during early studies of human nutrition, exemplified by vitamin B1 (thiamine), vitamin B2 (riboflavin), vitamin B3 (vitamin P or vitamin PP, or niacin), vitamin B5 (pantothenic acid), vitamin B6 (pyridoxine and pyridoxamine), vitamin B7 (vitamin H, vitamin B-w, or biotin), vitamin B9 (vitamin M, vitamin B-c, or folic acid), vitamin B12 (cyanocobalamin).

A B vitamin molecule also includes without limitation, “nonhuman forms” discovered by study of nutrition in other life form (animals, bacteria, yeast, etc.) such as vitamin B4 (adenine), vitamin B8 (ergadenylic acid), vitamin B10 (para-aminobenzoic acid), vitamin B11 (salicylic acid or vitamin S), vitamin B13 (pyrimidinecarboxylic acid or orotic acid), vitamin B14 (a mixture of vitamin B10 and vitamin B11), vitamin B15 (pangamic acid or dimethylglycine), vitamin B16, vitamin B17 (amygdalin), vitamin B22, vitamin B-t (L-carnitine), and vitamin B-x (para-aminobenzoic acid).

The B vitamins often work together to deliver a number of health benefits to the body, such as, bolstering metabolism, maintaining healthy skin and muscle tone, enhancing immune and nervous system function, and promoting cell growth and division, including that of the red blood cells, that are important at threshold minimum levels to prevent development of anemia. Combined, the B vitamins assist in combating the symptoms and causes of stress, depression, and cardiovascular disease. B vitamins are water soluble and are dispersed throughout the body and must be replenished daily, as any excess is excreted generally in the urine.

A “vitamin B2 molecule”, as used herein, refers to any or all of vitamin B2, riboflavin or vitamin G. As used herein, this term includes also the coenzyme forms, flavin adenine dinucleotide (FAD) and flavin adenine mononucleotide (FMN). B2 molecules are easily absorbed, water-soluble micronutrients that support energy production by aiding in the metabolism of fats, carbohydrates, and proteins. Vitamin B2 molecules are also needed for red blood cell formation and respiration, antibody production, and for regulating human growth and reproduction. They function as antioxidants by scavenging damaging particles in the body known as free radicals. Vitamin B2 molecules are important for healthy skin, nails, hair growth and general good health, including regulating thyroid activity.

Vitamin B2 deficiency manifests itself as cracks and sores at the corners of the mouth, eye disorders, inflammation of the mouth and tongue, skin lesions, dermatitis, dizziness, hair loss, insomnia, light sensitivity, poor digestion, retarded growth, and the sensation of burning feet.

An exemplary structure of the vitamin B2 molecule is shown below:

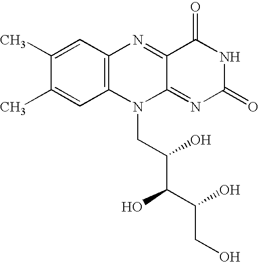

A “vitamin B3 molecule”, as used herein, refers to any or all of vitamin B3, niacin, or nicotinic acid. These include the amide form, nicotinamide or niacinamide. Vitamin B3 molecules are water-soluble vitamins whose derivatives such as NADH, NAD, NAD⁺, and NADP play important roles in energy metabolism in the living cell and DNA repair. These molecules also assist the body make various sex and stress-related hormones in the adrenal glands and other parts of the body. A vitamin B3 molecule is effective in improving circulation and reducing cholesterol levels in the blood.

Lack of a vitamin B3 molecule causes the deficiency disease pellagra. A mild B3 deficiency causes a slow down of the metabolism, which in turn causes a decrease in cold tolerance and is a potential contributing factor towards obesity.

In vivo synthesize a vitamin B3 molecule is initiated from the 5-membered aromatic heterocycle of the amino acid tryptophan, which is cleaved and rearranged with the alpha amino group of tryptophan into the 6-membered aromatic heterocycle of a vitamin B3 molecule. The reaction proceeds as follows: tryptophan->kynurenine->3-hydroxy kynurenine (B6 enzyme needed)->vitamin B3 molecule. The liver can synthesize vitamin B3 molecules from the amino acid tryptophan, and the synthesis is slow and requires vitamin B6, i.e., 60 mg of tryptophan are required to make one milligram of a vitamin B3 molecule.

An exemplary structure of the vitamin B3 molecule is shown below:

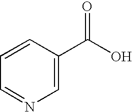

A “vitamin B6 molecule”, as used herein, refers to any or all of vitamin B6, pyridoxine, pyridoxal, and pyridoxamine. These molecules are converted to pyridoxal 5′-phosphate (PLP) in the liver. PLP is an important cofactor for numerous metabolic enzymes, such as aminotransferases, amino acid racemases, and amino acid decarboxylases, most of which have amino group-containing compounds as substrates. In the absence of PLP, a substantial number of cellular biosynthetic and catabolic pathways would cease to function.

Two pathways of de novo PLP synthesis are known, the PdxA/PdxJ pathway and the PDX1/PDX2 pathway. Organisms appear to contain either one or the other pathway of de novo PLP synthesis. Vitamin B6 comprises, in addition to PLP, precursors of PLP in phosphorylated and non-phosphorylated forms, and these compounds are referred to as B6 vitamers. Non-phosphorylated vitamers pyridoxine, pyridoxal and pyridoxamine can be taken up by many bacteria, fungi, plants, and mammalian cells and converted into PLP by a salvage pathway.

An exemplary structure of the vitamin B6 molecule is shown below:

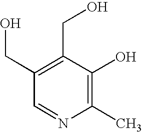

A “vitamin B9 molecule”, as used herein, refers to any or all vitamin B9, folic acid and folate. The B9 molecule is a water-soluble vitamin that is important for the production and maintenance of new cells, particularly during periods of rapid cell division and growth such as infancy and pregnancy. The B9 molecule is needed to replicate DNA and synthesize RNA, and is involved in the synthesis, repair, and functioning of DNA. A deficiency of folate may result in damage to DNA that may lead to cancer. Both adults and children need vitamin B9 molecules to make normal red blood cells and prevent anemia.

In the form of a series of tetrahydrofolate compounds, folate derivatives are coenzymes in a number of single carbon transfer reactions biochemically, and also is involved in the synthesis of dTMP (2′-deoxythymidine-5′-phosphate) from dUMP (2′-deoxyuridine-5′-phosphate).

The pathway in the formation of tetrahydrofolate (Fe) is the reduction of folate (F) to dihydrofolate (FH₂) by folate reductase, and then the subsequent reduction of dihydrofolate to tetrahydrofolate (FH₄) by dihydrofolate reductase. Methylene tetrahydrofolate (CH₂FH₄) is formed from tetrahydrofolate by the addition of methylene groups from one of three carbon donors: formaldehyde, serine, or glycine. Methyl tetrahydrofolate (CH₃—FH₄) can be made from methylene tetrahydrofolate by reduction of the methylene group, and formyl tetrahydrofolate (CHO—FH₄, folinic acid) is made by oxidation of the methylene tetrahydrofolate.

Signs of vitamin B9 deficiency include diarrhea, loss of appetite, weight loss, weakness, sore tongue, headaches, heart palpitations, irritability, and behavioral disorders. In adults, anemia is a sign of advanced vitamin B9 deficiency. In infants and children, vitamin B9 deficiency can slow growth rate.

An exemplary structure the vitamin B9 molecule is shown below:

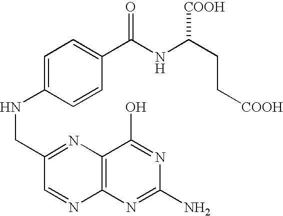

A “vitamin B12 molecule”, as used herein, refers to any or all of a group of cobalt containing tetrapyrrole compounds known as corrinoids. Examples include, cobalamin, cyanocobalamin, hydroxocobalamin, and thiocyanate cobalamin. The structure of vitamin B12 molecules comprises a nucleotide (base, ribose and phosphate) attached to a corrin ring which is made up of four pyrrole groups and an atom of cobalt in the center. The cobalt atom bonds to a methyl group, a deoxyadenosyl group, and a hydroxyl group or a cyano group. A vitamin B12 molecule includes the coenzyme forms of vitamin B12, i.e., methylcobalamin and 5-deoxyadenosylcobalamin (adenosylcobalamin).

A vitamin B12 molecule also includes any vitamin B12 precursor having vitamin B12 activity as detectable in the turbidimetric bioassay based on the growth response of Lactobacillus leichmanii ATCC 7830 as described in detail in the United States Pharmacopoeia, The National Formulary, 1995, pp. 1719-1721, United States Pharmacopoeial Convention, Inc., Rockville, Md. Examples of such precursors include cobyrinic acid, uroporphyrinogen III, hydrogenobryinic acid, precorrin-3, and precorrin-6x. Further examples of vitamin B12 precursors are described in detail in Thibaut et al., 1990 Proc. Natl. Acad. Sci. 87:8795-8799.

A vitamin B12 molecule further includes any vitamin B12 analog or derivative. An example of a vitamin B12 analog or derivative is a vitamin B12 molecule in which the alpha-ribose moieties of the nucleotide ligand are succinylated; another example is a vitamin B12 molecule lacking an axial nucleotide, and the molecule is further substituted with one or more alkyl halide groups.

Deficiency of vitamin B12 results in hematological, neurological and gastrointestinal effects. The hematological effects are caused by interference with DNA synthesis. The hematologic symptoms and signs of vitamin B12 deficiency, include hypersegmentation of polymorphonuclear leukocytes, macrocytic, hyperchromic erythrocytes, elevated mean corpuscular volume (MCV), elevated mean corpuscular hemoglobin concentration (MCH, MCHC), a decreased red blood cell count, pallor of the skin, decreased energy and easy fatigability, shortness of breath and palpitations.

The neurological effects of the vitamin B12 deficiency include tingling and numbness in the extremities (particularly the lower extremities), loss of vibratory and position sensation, abnormalities of gait, spasticity, Babinski's responses, irritability, depression and cognitive changes (loss of concentration, memory loss, dementia). Visual disturbances, impaired bladder and bowel control, insomnia and impotence may also occur.

Gastrointestinal effects of vitamin B12 deficiency include intermittent diarrhea and constipation, abdominal pain, flatulence and burning of the tongue (glossitis). Anorexia and weight loss are general symptoms of vitamin B12 deficiency.

Pathologies or defects can reduce efficiency or function of this pathway, such as an autoimmune condition involving formation of antibodies against the cells producing intrinsic factor; presence of a fish tapeworm; or the after-effects of surgery to the small intestine which results in the surface of the small intestine being insufficient to obtain B12 and intrinsic factor. These pathologies or defects result in less efficient absorption of vitamin B12, and could be ameliorated by administration of a higher dosage of vitamin B12.

An exemplary structure of a vitamin B12 molecule is shown below:

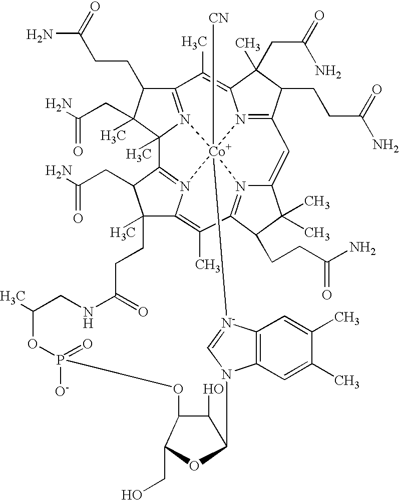

Use of a B Vitamin Molecule with an HDAC Inhibitor

Myelosuppression is a condition in which bone marrow activity is decreased, resulting in fewer blood cells (produced in bone marrow), for example, anemia (low red blood cells), thrombocytopenia (low platelets) and leucopenia (low white blood cells). In general, myelosuppression, especially thrombocytopenia, is a common dose-limiting toxicity side effect for most anti-cancer agents. This toxicity can interfere with effective cancer chemotherapy and lead to a delay in subsequent courses and/or reduction in treatment dose. Severe myelosuppression can lead to infection due to prolonged inhibition of the host-defense mechanisms involving white blood cells.

Without being limited by any particular theory or mechanism of action, the action of the B vitamins in vivo promotes essential cell division and cell replication pathways. For example, vitamin B12 and vitamin B9 are involved in the process of rapid synthesis of DNA during cell division, particularly in the synthesis of the building blocks for DNA and RNA synthesis. Vitamin B3 is involved in repair of DNA and vitamin B2 is involved in synthesis of red blood cells.

These processes are especially important in tissues where cells are dividing rapidly, particularly the bone marrow tissues responsible for red blood cell formation. An insufficient amount of the B vitamins results in decreased availability of essential building blocks, such as thymidylic acid and purine nucleotides, precursors of DNA synthesis which are necessary for normal cell division.

Without being limited by any particular theory or mechanism of action, side effects in a patient resulting from a round of chemotherapy treatment, typically described as, cracks and sores at the corners of the mouth, skin lesions, dermatitis, hair loss, poor digestion, decrease in cold tolerance, diarrhea, loss of appetite, weight loss, weakness, headaches, anemia (low red blood cell count), thrombocytopenia (low platelet count), leucopenia (low white blood cell count), pallor of the skin, decrease in energy, easy fatigability, and abdominal pain, are similar to conditions which are manifest by a person having B vitamin deficiencies.

B vitamin Dosage

A B vitamin molecule is administered systemically, for example, orally, subcutaneously, intramuscularly, and intravenously. The dose of B vitamin administered depends on form and route of delivery, i.e., injection, nasal gel, or oral administration by lozenges or by sublingual tablets, as is well known to one of ordinary skill in the art of nutritional supplementation. As B vitamins are water soluble and as excess is not stored but is excreted generally in the urine, it is important that they are replenished daily. Table 3 below shows typical dosages of different B vitamins.

TABLE 3
Dosages of different B vitamins

| | Minimum | Typical | | Potential side |
|---|---|---|---|---|
| | recommended daily | therapeutic daily | Upper limit of | effects |
| Vitamin | allowance (MDR) | dose | daily intake | at upper limits |
| B2 | 1 mg-2 mg | 50 mg-100 mg | N/A | No known |
| | | | | toxicity |
| B3 | 15 mg-25 mg | 100 mg-500 mg | Above 3000 mg | Liver problems |
| B6 | 1.5 mg-2.5 mg | 50 mg-100 mg | Above 100 mg | Numbness and |
| | | | | tingling in the |
| | | | | fingers and toes |
| B9 | 350 μg-450 μg | 500 μg-1000 μg | N/A | No known |
| | | | | toxicity |
| B12 | 3 μg-30 μg | 500 μg-5000 μg | N/A | No known |
| | | | | toxicity |

The amount of total absorption of these B vitamins increases with increased intake. Without being limited by any particular theory or mechanism of action, higher doses than the minimum daily requirement are beneficial under circumstances of vitamin stress, such as during anti-cancer chemotherapy. Excess amounts of B vitamins that are administered are subsequently excreted in the feces and in the urine. In general, if the circulating levels of the B vitamins exceed the B vitamin binding capacity of the blood, the excess is excreted in the urine.

### Pharmaceutical Compositions

The HDAC inhibiting compounds and molecules of the pharmaceutical compositions, uses, and methods described above are often used in the form of a pharmaceutically acceptable salt. Pharmaceutically acceptable salts include, when appropriate, pharmaceutically acceptable base addition salts and acid addition salts, for example, metal salts, such as alkali and alkaline earth metal salts, ammonium salts, organic amine addition salts, and amino acid addition salts, and sulfonate salts. Acid addition salts include inorganic acid addition salts such as hydrochloride, sulfate and phosphate, and organic acid addition salts such as alkyl sulfonate, arylsulfonate, acetate, maleate, fumarate, tartrate, citrate and lactate. Examples of metal salts are alkali metal salts, such as lithium salt, sodium salt and potassium salt, alkaline earth metal salts such as magnesium salt and calcium salt, aluminum salt, and zinc salt. Examples of ammonium salts are ammonium salt and tetramethylammonium salt. Examples of organic amine addition salts are salts with morpholine and piperidine. Examples of amino acid addition salts are salts with glycine, phenylalanine, glutamic acid and lysine. Sulfonate salts include mesylate, tosylate and benzene sulfonic acid salts.

The invention provides also pharmaceutical compositions comprising an HDAC inhibiting compound and a B vitamin molecule and their use in the therapeutic (in a broader aspect of the invention also prophylactic) treatment or a method of treatment of an HDAC dependent disease, including, for example, the diseases mentioned above, to the HDAC inhibiting compounds for the use and to the preparation of pharmaceutical preparations, for the uses.

The present invention also provides pro-drugs of the HDAC inhibiting compounds that convert in vivo to the HDAC inhibiting compounds of the present methods as such, and a B vitamin molecule. Any reference to an HDAC inhibiting compound of the present methods is therefore to be understood as referring also to the corresponding pro-drugs of the HDAC inhibiting compounds as appropriate and expedient.

The HDAC inhibiting compounds herein may be used, for example, for the preparation of pharmaceutical compositions that comprise an effective amount of an HDAC inhibitor herein and a B vitamin molecule, or a pharmaceutically acceptable salt thereof, as active ingredient together or in admixture with a significant amount of one or more inorganic or organic, solid or liquid, pharmaceutically acceptable carriers.

The compositions herein are suitable for administration to a warm-blooded animal, including, for example, a human (or to cells or cell lines derived from a warm-blooded animal, including for example, a human cell, e.g., lymphocytes), for the treatment or, in another aspect of the invention, prevention of (also referred to as prophylaxis against) a disease that responds to inhibition of HDAC activity, comprising an amount of a compound of the present methods or a pharmaceutically acceptable salt thereof, which is effective for this inhibition, including the inhibition of activity of an HDAC or inhibition of an HDAC protein interacting with another transcriptional effector protein, together with at least one pharmaceutically acceptable carrier, and a B vitamin molecule.

The pharmaceutical compositions according to the methods are those for enteral, such as nasal, rectal or oral, or parenteral, such as intramuscular or intravenous, administration to warm-blooded animals (including, for example, a human), that comprise an effective dose of the pharmacologically active ingredient, alone or together with a significant amount of a pharmaceutically acceptable carrier. The dose of the active ingredient depends on the species of warm-blooded animal, the body weight, the age and the individual condition, individual pharmacokinetic data, the disease to be treated and the mode of administration.

The dose of a HDAC inhibitor of the present methods or a pharmaceutically acceptable salt thereof to be administered to warm-blooded animals, for example humans of approximately 70 kg body weight, is for example, from approximately 3 mg to approximately 10 g, from approximately 10 mg to approximately 1.5 g, from about 100 mg to about 1000 mg/person/day, divided into 1-3 single doses which may, for example, be of the same size. Usually, children receive half of the adult dose.

The dose of the B vitamin molecule to be administered to warm-blooded animals, for example humans of approximately 70 kg body weight, is for example at least about 50 micrograms (μg), at least about 80 μg, 90 μg, 100 μg, or at least about 500 μg, at least about 25 milligrams (mg), 30 mg, 40 mg, or at least about 50 mg, to at least about 500 mg.

The pharmaceutical compositions have from approximately, for example, 1% to approximately 95%, or from approximately 20% to approximately 90%, active ingredients. Pharmaceutical compositions according to the invention may be, for example, in unit dose form, such as in the form of ampoules, vials, suppositories, dragées, tablets or capsules.

The pharmaceutical compositions of the present invention are prepared in a manner known per se, for example by means of conventional dissolving, lyophilizing, mixing, granulating or confectioning processes.

Solutions of the active ingredients, and also suspensions, and especially isotonic aqueous solutions or suspensions, are used, it being possible, for example in the case of lyophilized compositions that have the active ingredient alone or together with a carrier, for example mannitol, for such solutions or suspensions to be produced prior to use. The pharmaceutical compositions may be sterilized and/or may comprise excipients, for example preservatives, stabilizers, wetting and/or emulsifying agents, solubilizers, salts for regulating the osmotic pressure and/or buffers, and are prepared in a manner known per se, for example by means of conventional dissolving or lyophilizing processes. The solutions or suspensions may have viscosity-increasing substances, such as sodium carboxymethylcellulose, carboxymethylcellulose, dextran, polyvinylpyrrolidone or gelatin.

Suspensions in oil comprise as the oil component the vegetable, synthetic or semi-synthetic oils customary for injection purposes. There may be mentioned, for example, liquid fatty acid esters that contain as the acid component a long-chained fatty acid having from 8-22, or from 12-22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brasidic acid or linoleic acid, if desired with the addition of antioxidants, for example vitamin E, β-carotene or 3,5-di-tert-butyl-4-hydroxytoluene. The alcohol component of those fatty acid esters has a maximum of 6 carbon atoms and is a mono- or poly-hydroxy, for example a mono-, di- or tri-hydroxy, alcohol, for example methanol, ethanol, propanol, butanol or pentanol or the isomers thereof, but especially glycol and glycerol. The following examples of fatty acid esters are therefore to be mentioned: ethyl oleate, isopropyl myristate, isopropyl palmitate, “Labrafil M 2375” (polyoxyethylene glycerol trioleate, Gattefossé, Paris), “Miglyol 812” (triglyceride of saturated fatty acids with a chain length of C8 to C12, Hüls AG, Germany), but especially vegetable oils, such as cottonseed oil, almond oil, olive oil, castor oil, sesame oil, soybean oil and more especially groundnut oil.

The injection compositions are prepared in customary manner under sterile conditions; the same applies also to introducing the compositions into ampoules or vials and sealing the containers.

Pharmaceutical compositions for oral administration can be obtained by combining the active ingredients with solid carriers, if desired granulating a resulting mixture, and processing the mixture, if desired or necessary, after the addition of appropriate excipients, into tablets, dragée cores or capsules. It is also possible for them to be incorporated into plastics carriers that allow the active ingredients to diffuse or be released in measured amounts.

Suitable carriers are for example, fillers, such as sugars, for example lactose, saccharose, mannitol or sorbitol, cellulose preparations and/or calcium phosphates, for example tricalcium phosphate or calcium hydrogen phosphate, and binders, such as starch pastes using for example corn, wheat, rice or potato starch, gelatin, tragacanth, methylcellulose, hydroxypropylmethylcellulose, sodium carboxymethylcellulose and/or polyvinylpyrrolidone, and/or, if desired, disintegrators, such as the above-mentioned starches, and/or carboxymethyl starch, crosslinked polyvinylpyrrolidone, agar, alginic acid or a salt thereof, such as sodium alginate. Excipients are especially flow conditioners and lubricants, for example silicic acid, talc, stearic acid or salts thereof, such as magnesium or calcium stearate, and/or polyethylene glycol. Dragée cores are provided with suitable, optionally enteric, coatings, there being used, inter alia, concentrated sugar solutions which may comprise gum arabic, talc, polyvinylpyrrolidone, polyethylene glycol and/or titanium dioxide, or coating solutions in suitable organic solvents, or, for the preparation of enteric coatings, solutions of suitable cellulose preparations, such as ethylcellulose phthalate or hydroxypropylmethylcellulose phthalate. Capsules are dry-filled capsules made of gelatin and soft sealed capsules made of gelatin and a plasticizer, such as glycerol or sorbitol. The dry-filled capsules may comprise the active ingredients in the form of granules, for example with fillers, such as lactose; binders, such as starches, and/or glidants, such as talc or magnesium stearate, and if desired with stabilizers. In soft capsules the active ingredients are preferably dissolved or suspended in suitable oily excipients, such as fatty oils, paraffin oil or liquid polyethylene glycols, it being possible also for stabilizers and/or antibacterial agents to be added. Dyes or pigments may be added to the tablets or dragée coatings or the capsule casings, for example for identification purposes or to indicate different doses of active ingredient.

### Pharmaceutical Composition Including a Combination of an HDAC and a B Vitamin Molecule

The present invention provides pharmaceutical compositions containing an HDAC inhibitor and a B vitamin molecule, the composition being suitable for administration to a subject, for example, a human, for the treatment, prevention or amelioration of a disease that responds to inhibition of HDAC activity, especially a proliferative disease.

The pharmaceutical compositions generally include an effective dose of each of the HDAC inhibitor and the B vitamin molecule. As used herein, an “effective dose” means an amount of each active component that is different from an optimal amount of that component if administered in a therapeutic regiment absent the other active component. An effective dose of the pharmaceutical composition when administered to a subject, prevents or ameliorates a disease symptom, i.e. reduces and/or ameliorates the proliferative disorder or HDAC dependent disease or tumor, cell mass or target cell and also produces fewer side effects compared to these symptoms in a control subject administered either the HDAC inhibitor or the B vitamin molecule alone. One of ordinary skill in the art of treatment of proliferative diseases can readily determine an effective amount of each component in the combination. For example, side effects are prevented or ameliorated by the presence in the combination of a particular dose of B vitamin, then a greater amount of the HDAC inhibitor component can be included in the pharmaceutical composition to be administered to the subject, compared to a control amount, which is the amount of the HDAC inhibitor alone that would be administered to the subject. It is an object of the methods and compositions herein that in the presence or co-administration of a B vitamin, an effective dose of an HDAC inhibitor is reduced compared to an effective dose in the absence of a B vitamin, due to increased efficacy of these compounds in the presence of the B vitamin.

Anti-tumor agents are often limited in dose by undesirable side effects, hence efficacy is limited by the choice of the dose, based on a subject's ability to tolerate that dose. Side effects include, e.g., thrombocytopenia and anemia and other conditions that result from inhibition by the agent of hematopoiesis. B vitamin molecules are here surprisingly found to prevent or ameliorate these effects, hence a higher dosage of the HDAC inhibitor is tolerated in a therapeutic regimen with the pharmaceutical composition herein that includes also an amount of B vitamin molecules. Starting with administration of a standard amount of the HDAC inhibitor to an experimental subject, such as a mouse, B vitamin molecules are administered in varying amounts to each mouse in each experimental group, except for the control group which is administered the HDAC inhibitor alone, or the control is administered neither agent. Symptoms of both remediation of the disease and the side effects are monitored, by any cancer assay, and by a convenient assay of side effects, such as blood clotting time, red blood cell amount, etc. Then starting with a B vitamin molecule dose that prevents or ameliorates symptoms, new groups of mice are tested for tolerance of even greater doses of HDAC, in combination with increasing doses of B vitamin until the effective doses of the combination having greatest efficacy with fewest side effects are determined, by these routine procedures without undue experimentation.

A decrease in proliferation of the tumor, cell mass or target cell, or decreased metastasis, can be analyzed by observing a decrease in, for example, tumor size, number of metastases, tumor necrosis, cell proliferation rate, or cell apoptosis. As is standard in the pharmaceutical arts, effective dose of the pharmaceutical composition depends on the species of warm-blooded animal, the body weight, the age and the individual condition, individual pharmacokinetic data, the disease to be treated and the mode of administration.

Under certain conditions, the B vitamin molecules have a synergistic effect in combination with a class of HDAC inhibitors, in preventing or ameliorating a disease that responds to inhibition of HDAC activity, e.g., a proliferative disease. Thus the pharmaceutical composition includes an effective dose which is a lesser amount of the HDAC inhibitor component, compared to administering to the subject the HDAC inhibitor alone, to obtain a comparable therapeutic effect.

An effective dose of the B vitamin component of the pharmaceutical composition is an amount that prevents or ameliorates one or more side effects resulting from administration of an HDAC inhibitor, and is described herein.

One of ordinary skill in the art of treatment of proliferative diseases can readily determine the extent to which a side effect of a proliferative disease or from treatment of the disease, results from a specific vitamin deficiency. Under certain conditions, an effective dose of the B vitamin component of the pharmaceutical composition is an amount which prevents or ameliorates the vitamin deficiency present in the subject with the proliferative disease thereby further reducing side effects of the HDAC inhibitor.

### Combinations

An HDAC inhibiting compound of the present methods may also be used to advantage in combination with other antiproliferative agents. Such antiproliferative agents include, but are not limited to aromatase inhibitors; antiestrogens; topoisomerase I inhibitors; topoisomerase II inhibitors; microtubule active agents; alkylating agents; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; bisphosphonates; biological response modifiers; antiproliferative antibodies; heparanase inhibitors; inhibitors of Ras oncogenic isoforms; telomerase inhibitors; proteasome inhibitors; agents used in the treatment of hematologic malignancies; compounds which target, decrease or inhibit the activity of Flt-3; Hsp90 inhibitors; temozolomide (TEMODAL®); and leucovorin.

The phrase, “aromatase inhibitor” as used herein relates to a compound which inhibits the estrogen production, i.e., the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane can be administered, e.g., in the form as it is marketed, e.g., under the trademark AROMASIN. Formestane can be administered, e.g., in the form as it is marketed, e.g., under the trademark LENTARON. Fadrozole can be administered, e.g., in the form as it is marketed, e.g., under the trademark AFEMA. Anastrozole can be administered, e.g., in the form as it is marketed, e.g., under the trademark ARIMIDEX. Letrozole can be administered, e.g., in the form as it is marketed, e.g., under the trademark FEMARA or FEMAR. Aminoglutethimide can be administered, e.g., in the form as it is marketed, e.g., under the trademark ORIMETEN. A combination of the invention comprising a chemotherapeutic agent which is an aromatase inhibitor is particularly useful for the treatment of hormone receptor positive tumors, e.g., breast tumors.

The term “antiestrogen” as used herein relates to a compound that antagonizes the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g., under the trademark NOLVADEX. Raloxifene hydrochloride can be administered, e.g., in the form as it is marketed, e.g., under the trademark EVISTA. Fulvestrant can be formulated as disclosed in U.S. Pat. No. 4,659,516 or it can be administered, e.g., in the form as it is marketed, e.g., under the trademark FASLODEX. A combination of the invention comprising a chemotherapeutic agent which is an antiestrogen is particularly useful for the treatment of estrogen receptor positive tumors, e.g., breast tumors.

The term “anti-androgen” as used herein relates to any substance which is capable of inhibiting the biological effects of androgenic hormones and includes, but is not limited to, bicalutamide (CASODEX), which can be formulated, e.g., as disclosed in U.S. Pat. No. 4,636,505.

The phrase, “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g., under the trademark ZOLADEX. Abarelix can be formulated, e.g., as disclosed in U.S. Pat. No. 5,843,901.

The phrase, “topoisomerase I inhibitor” as used herein includes, but is not limited to topotecan, gimatecan, irinotecan, camptothecan and its analogues, 9-nitrocamptothecin and the macromolecular camptothecin conjugate PNU-166148 (compound A1 in WO99/17804). Irinotecan can be administered, e.g., in the form as it is marketed, e.g., under the trademark CAMPTOSAR. Topotecan can be administered, e.g., in the form as it is marketed, e.g., under the trademark HYCAMTIN.

The phrase, “topoisomerase II inhibitor” as used herein includes, but is not limited to the anthracyclines such as doxorubicin (including liposomal formulation, e.g., CAELYX), daunorubicin, epirubicin, idarubicin and nemorubicin, the anthraquinones mitoxantrone and losoxantrone, and the podophyllotoxins etoposide and teniposide. Etoposide can be administered, e.g., in the form as it is marketed, e.g., under the trademark ETOPOPHOS. Teniposide can be administered, e.g., in the form as it is marketed, e.g., under the trademark VM 26-BRISTOL. Doxorubicin can be administered, e.g., in the form as it is marketed, e.g., under the trademark ADRIBLASTIN or ADRIAMYCIN. Epirubicin can be administered, e.g., in the form as it is marketed, e.g., under the trademark FARMORUBICIN. Idarubicin can be administered, e.g., in the form as it is marketed, e.g., under the trademark ZAVEDOS. Mitoxantrone can be administered, e.g., in the form as it is marketed, e.g., under the trademark NOVANTRON.

The phrase, “microtubule active agent” relates to microtubule stabilizing, microtubule destabilizing agents and microtublin polymerization inhibitors including, but not limited to taxanes, e.g., paclitaxel and docetaxel, vinca alkaloids, e.g., vinblastine, including vinblastine sulfate, vincristine including vincristine sulfate, and vinorelbine, discodermolides, cochicine and epothilones and derivatives thereof, e.g., epothilone B or D or derivatives thereof. Paclitaxel may be administered e.g., in the form as it is marketed, e.g., TAXOL. Docetaxel can be administered, e.g., in the form as it is marketed, e.g., under the trademark TAXOTERE. Vinblastine sulfate can be administered, e.g., in the form as it is marketed, e.g., under the trademark VINBLASTIN R.P. Vincristine sulfate can be administered, e.g., in the form as it is marketed, e.g., under the trademark FARMISTIN. Discodermolide can be obtained, e.g., as disclosed in U.S. Pat. No. 5,010,099. Also included are Epothilone derivatives which are disclosed in WO 98/10121, U.S. Pat. No. 6,194,181, WO 98/25929, WO 98/08849, WO 99/43653, WO 98/22461 and WO 00/31247. Included are Epothilone A and/or B.

The phrase, “alkylating agent” as used herein includes, but is not limited to, cyclophos-phamide, ifosfamide, melphalan or nitrosourea (BCNU or Gliadel). Cyclophosphamide can be administered, e.g., in the form as it is marketed, e.g., under the trademark CYCLOSTIN. Ifosfamide can be administered, e.g., in the form as it is marketed, e.g., under the trademark HOLOXAN.

The phrase, “histone deacetylase inhibitors” or “HDAC inhibitors” relates to compounds which inhibit at least one example of the class of enzymes known as a histone deacetylase, as described herein, and which compounds generally possess antiproliferative activity. Previously disclosed HDAC inhibitors include compounds disclosed in, e.g., WO 02/22577, including N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(2-methyl-1H-indol-3-yl)-ethyl]-amino]methyl]phenyl]-2E-2-propenamide and pharmaceutically acceptable salts thereof. It further includes Suberoylanilide hydroxamic acid (SAHA). Other publicly disclosed HDAC inhibitors include butyric acid and its derivatives, including sodium phenylbutyrate, thalidomide, trichostatin A and trapoxin.

Further HDAC inhibitors include compounds such as hydroxamic acids, hydroxamates, hydroxyamides, cyclic peptides, benzamides, benzimidazoles, short-chain fatty acids, mercaptomides, carbamic acids, carbonyls, piperazinyls, piperidinyls, morpholinyls, sulfonyls, amines, amides, valproic acids, oximes, dioxanes, epoxides, lactams, and depudecin.

Examples of the above HDAC inhibitors are found in U.S. Pat. Nos. 6,831,061 (Lee et al.); 6,800,638 (Georges et al.); 6,399,568 (Nishino et al.); 6,124,495 (Neiss et al.); and 5,939,455 (Rephaeli), and patent applications: WO03082288 (Watkins et al.); CA2520611 (Miller et al.); WO2005075466 (Bordogna et al.); WO2005053610 (Miller et al.); US2005124679 (Kim et al.); WO2005014588 (Dyke et al.); US2006058553 (Leahy et al.); WO2005097770 (Setti); WO2005058803 (LeBlond et al.); WO2005040161 (Stunkel et al.); WO2006025683 (Lee et al.); WO2006016680 (Ishibashi et al.); WO2004072047 (Urano et al.); WO2006028972 (Ahmed et al.); WO2005075446 (Koyama et al.); US2006058282 (Finn et al.); US2005143385 (Watkins et al.); EP1635800 (Wash et al.); US2005148613 (Van Emelen et al.); WO03099760 (Lan-Hargest et al.); WO03099789 (Lan-Hargest et al.); ZA200407237 (Van Emelen et al.); WO2006010749 (Van Brandt et al.); WO03076401 (Van Emelen et al.); US2006030543 (Malecha et al.); WO2005040101 (Lim et al.); WO2006010750 (Verdonck et al.); US2005119250 (Angibaud et al.); US2004157841 (Fertig et al.); US2004162317 (Fertig et al.); WO2006005955 (Chakravarty et al.); WO2006005941 (Chakravarty et al.); WO2005065681 (Bressi et al.); WO03070691 (Uesato et al.); US2005038113 (Groner et al.); CA2519301 (Fertig et al.); WO02089782 (Schreiber et al.); US2005282890 (Zheng); WO03099272 (Lan-Hargest et al.); US2004077698 (Georges et al.); US2002120099 (Basting); U.S. Pat. No. 6,656,905 (Mori et al.); and U.S. Pat. No. 6,399,568 (Nishino et al.); HK1079042; US2005171103 (Stokes et al.); HK1046277 (Ishibashi et al.); US2006069157 (Ferrante); WO2005055928 (Chen et al.); and WO9800127 (Rephaeli et al.).

The term “antineoplastic antimetabolite” includes, but is not limited to, 5-Fluorouracil or 5-FU, capecitabine, gemcitabine, DNA demethylating agents, such as 5-azacytidine and decitabine, methotrexate and edatrexate, and folic acid antagonists such as pemetrexed. Capecitabine can be administered, e.g., in the form as it is marketed, e.g., under the trademark XELODA. Gemcitabine can be administered, e.g., in the form as it is marketed, e.g., under the trademark GEMZAR. Also included is the monoclonal antibody trastuzumab which can be administered, e.g., in the form as it is marketed, e.g., under the trademark HERCEPTIN.

The phrase, “platin compound” as used herein includes, but is not limited to, carboplatin, cis-platin, cisplatinum and oxaliplatin. Carboplatin can be administered, e.g., in the form as it is marketed, e.g., under the trademark CARBOPLAT. Oxaliplatin can be administered, e.g., in the form as it is marketed, e.g., under the trademark ELOXATIN.

The phrase, “compounds targeting/decreasing an HDAC activity; or a histone deacetylase activity; or further anti-angiogenic compounds” as used herein includes, but is not limited to: HDAC1-11 inhibitors, e.g.: HDAC2, HDAC3 AND HDAC8 inhibitors.

The following list of proteins involved in signal transduction illustrates far reaching effects of modulating transcription by inhibiting HDAC activity:

i) compounds targeting, decreasing or inhibiting the activity of the platelet-derived growth factor-receptors (PDGFR), such as compounds which target, decrease or inhibit the activity of PDGFR, especially compounds which inhibit the PDGF receptor, e.g., a N-phenyl-2-pyrimidine-amine derivative, e.g., imatinib, SU101, SU6668, and GFB-111;

ii) compounds targeting, decreasing or inhibiting the activity of the fibroblast growth factor-receptors (FGFR);

iii) compounds targeting, decreasing or inhibiting the activity of the insulin-like growth factor receptor I(IGF-IR), such as compounds which target, decrease or inhibit the activity of IGF-IR, especially compounds which inhibit the IGF-IR receptor, such as those compounds disclosed in WO 02/092599; and/or

iv) compounds targeting, decreasing or inhibiting the activity of the c-Met receptor.

Tumor cell damaging approaches refer to approaches such as ionizing radiation. The phrase, “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See, e.g., Hellman, Principles of Radiation Therapy, Cancer, in Principles and Practice of Oncology, Devita et al., Eds., 4th Edition, Vol. 1, pp. 248-275 (1993).

The phrase, “EDG binders” as used herein refers a class of immunosuppressants that modulates lymphocyte recirculation, such as FTY720.

CERTICAN (everolimus, RAD) an investigational novel proliferation signal inhibitor that prevents proliferation of T-cells and vascular smooth muscle cells.

The phrase, “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives, such as PL-1, PL-2, PL-3, PL-4, PL-5, PL-6, PL-7 or PL-8 mentioned in Nandy et al., Acta Oncologica, Vol. 33, No. 8, pp. 953-961 (1994).

The phrase, “S-adenosylmethionine decarboxylase inhibitors” as used herein includes, but is not limited to the compounds disclosed in U.S. Pat. No. 5,461,076.

Also included are in particular those compounds, proteins or monoclonal antibodies of VEGF disclosed in WO 98/35958, e.g., 14-chloroanilino)-4-(4-pyridylmethyl)phthalazine or a pharmaceutically acceptable salt thereof, e.g., the succinate, or in WO 00/09495, WO 00/27820, WO 00/59509, WO 98/11223, WO 00/27819 and EP 0 769 947; those as described by Prewett et al, Cancer Res, Vol. 59, pp. 5209-5218 (1999); Yuan et al., Proc Natl Acad Sci USA, Vol. 93, pp. 14765-14770 (1996); Zhu et al., Cancer Res, Vol. 58, pp. 3209-3214 (1998); and Mordenti et al., Toxicol Pathol, Vol. 27, No. 1, pp. 14-21 (1999); in WO 00/37502 and WO 94/10202; ANGIOSTATIN, described by O'Reilly et al., Cell, Vol. 79, pp. 315-328 (1994); ENDOSTATIN, described by O'Reilly et al., Cell, Vol. 88, pp. 277-285 (1997); anthranilic acid amides; ZD4190; ZD6474; SU5416; SU6668; bevacizumab; or anti-VEGF antibodies or anti-VEGF receptor antibodies, e.g., rhuMAb and RHUFab, VEGF aptamer e.g., Macugon; FLT-4 inhibitors, FLT-3 inhibitors, VEGFR-2 IgG1 antibody, Angiozyme (RPI 4610) and Avastan.

Photodynamic therapy as used herein refers to therapy that uses certain chemicals known as photosensitizing agents to treat or prevent cancers. Examples of photodynamic therapy include treatment with agents, such as e.g., VISUDYNE and porfimer sodium.

The phrase, “angiostatic steroids” as used herein refers to agents which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to agents, such as e.g., fluocinolone, dexamethasone.

Other chemotherapeutic agents include, but are not limited to, plant alkaloids, hormonal agents and antagonists; biological response modifiers, preferably lymphokines or interferons; antisense oligonucleotides or oligonucleotide derivatives; or miscellaneous agents or agents with other or unknown mechanism of action.

The structure of the active agents identified by code numbers, generic or trade names may be taken from the actual edition of the standard compendium “The Merck Index” or from databases, e.g., Patents International (e.g., IMS World Publications).

The above-mentioned compounds, which can be used in combination with a compound of the present methods, can be prepared and administered as described in the art such as in the documents cited above.

A compound of the present methods may also be used to advantage in combination with known therapeutic processes, e.g., the administration of hormones or especially radiation.

A compound of the present invention may in also be used as a radiosensitizer, including, for example, the treatment of tumors which exhibit poor sensitivity to radiotherapy.

By the term “combination”, is meant either a fixed combination in one dosage unit form, or a kit of parts for the combined administration where a compound of the present invention and a combination partner may be administered independently at the same time or separately within time intervals that especially allow that the combination partners show a cooperative, e.g., synergistic, effect, or any combination thereof.

Example 1 below shows inhibition of tumor growth in rats in vivo, comparing administration of an HDAC inhibitor only with administration of a combination of the HDAC inhibitor and a B vitamin molecule.

Example 2 shows a comparison of the potency of single agent treatment using an HDAC inhibitor with combination therapy using the HDAC inhibitor and a B vitamin molecule. Table 3 shows the compounds and molecules and the concentrations of each that are administered to each group of animals.

Example 3 includes methods of assay for side effects from chemotherapeutic treatment. Table 4 shows the compounds and molecules and the concentrations of each that are administered to each group of animals.

The invention having been fully described, it is further exemplified by the following examples and claims, which are illustrative and are not meant to be further limiting. Those skilled in the art will recognize or be able to ascertain using no more than routine experimentation, numerous equivalents to the specific procedures described herein. Such equivalents are within the scope of the present invention and claims. The contents of all references, including issued patents and published patent applications, cited throughout this application are hereby incorporated herein by reference.

### EXAMPLES

The following protocols are provided to facilitate the practice of Examples 1-2.

### Drug Administration

For administration to rodents, HDAC inhibitors are complexed with 2-hydroxypropyl-β-cyclodextrin to increase solubility and allow for the HDAC inhibitor to be dissolved in water, as provided in Hockly et al., Proc Natl Acad Sci USA. 2003; 100(4): 2041-2046. Cyclodextrin and other HDAC inhibitor formulations are also be prepared as solid suspensions or dispersions. B vitamin molecules are dissolved in normal saline (0.9% NaCl).

### In Vivo Antitumor Testing

HCT116 colon carcinoma cell lines are used in assays and mouse xenograft models. Alternatively, B16-F10 murine melanoma cells are used. See U.S. patent application number US/2004/0229843 (Toole et al.). Tumors are propagated as subcutaneous injections of the cells into the appropriate rat recipient strain using HCT116 colon carcinoma cells or B16-F10 murine melanoma cells from donor mice.

The required number of animals are pooled at the start of the experiment prior to administration of an HDAC inhibitor alone or in combination with a B vitamin molecule for treatment of tumors, before distribution to the various treatment and control groups. Observations regarding efficacy and effects of potency on each animal is determined by assessing one or more parameters, such as tumor perfusion, tumor size, tumor number, or tumor weight, some of which are assayed on live animals throughout the course of the protocol, and others following termination and sacrifice of the animals.

Tumor size is determined by measurement of tumors with a caliper twice a week. Tumor weight (mg) is estimated from the formula: Tumor weight=(length×width2)/2. Tumor number is determined from autopsy data. Tumor perfusion is measured using the Evans blue dye uptake assay. Rats are administered Evans blue dye injected intravenously. The amount of Evans blue accumulated in the tumor is proportional to the blood flow through the tumor.

In general, compositions are administered orally (po), by intravenous delivery (iv) or subcutaneously (sc). Alternatively, ALZET pumps which are attainable from ALZA Corporation (Palo Alto, Calif.) are used. Formulations contain PBS or another vehicle, an HDAC inhibitor complexed with 2-hydroxypropyl-β-cyclodextrin and dissolved in water, or a B vitamin molecule dissolved in normal saline (0.9% NaCl) are injected sc under the skin in the dorsal region of the rat. On the day after injection, 0.5×10⁵ to 1.0×10⁶ tumor cells in 0.1 ml PBS are further injected immediately in the vicinity of the administrative site. Rats are euthanized by CO₂ after 14 day of treatment and tumor growth is assessed as described above.

### Example 1

### Improved Efficacy of a Combination Therapeutic Treatment of an HDAC Inhibitor and a B Vitamin Molecule

Single Agent Treatment with an HDAC Inhibitor

Administration of an HDAC inhibitor prior to subcutaneous implantation of B16-F10 murine melanoma cells inhibits growth of these cells. Extent of inhibition is related to relative time points of implanting the melanoma cells and administering the HDAC inhibitor. For each experiment, tumor cells are injected subcutaneously into animals to be treated in groups of 5 control and 5 experimental animals for each test condition.

Tumors are implanted into experimental animals, and growth of the tumors is monitored for about one-two weeks during which tumors increase in size. The HDAC inhibitor is then administered sc. The HDAC inhibitor is administered over a time course determined by the particular protocol, for example, a time course of 14 days. Control animals are administered vehicle (phosphate buffered solution (PBS) with a comparable amount of 2-hydroxypropyl-β-cyclodextrin as is used to complex with the HDAC inhibitor) only.

Administration of the HDAC inhibitor is found to inhibit tumor growth, i.e. is statistically correlated with reduction in one or more of tumor size, tumor weight, tumor number, and tumor perfusion, compared to data obtained on animals in the control group.

Combination Therapy with an HDAC Inhibitor and a B Vitamin Molecule

Synergistic antitumor activity is observed when the HDAC inhibitor is administered in combination with a B vitamin molecule in the treatment or prevention of HCT116 colon carcinoma tumors. In each experiment, tumor cells are injected subcutaneously into groups of 5 control and 5 experimental animals as above.

The HDAC inhibitor and the B vitamin molecule are administered either separately one day prior to injection of tumor cells, or are administered as a single solution in one injection. The HDAC inhibitor is administered adjacent to the site of implantation over the course of 14 days. The B vitamin molecule also is administered over the course of 14 days. Control animals receive vehicle only (PBS with a comparable amount of 2-hydroxypropyl-β-cyclodextrin as is used to complex with the HDAC inhibitor).

The combination of administration of the HDAC inhibitor and the B vitamin molecule is found to inhibit tumor growth, i.e. reduction in tumor size, tumor weight, tumor number, and tumor perfusion, compared to the control group. In comparison to the results obtained from administration of the single agent, i.e., only the HDAC inhibitor, the combination therapy of the HDAC inhibitor and the B vitamin molecule is found to more greatly inhibit tumor growth, i.e. reduction in tumor size, tumor weight, tumor number, and tumor perfusion.

### Example 2

### Efficacious Dose-Pharmacokinetics Determination for Potency Enhancement by a B Vitamin

Therapeutic treatment with a combination of an HDAC inhibitor and a B vitamin molecule is shown to produce therapeutic synergism with respect to inhibition of tumor appearance or growth, as shown in Example 1. A study is then conducted to compare the potency of single agent treatment using an HDAC inhibitor with combination therapy using varying amounts of the HDAC inhibitor and a constant amount of B vitamin molecule. Inhibition of tumor growth is analyzed by measuring tumor size, tumor weight, tumor number and tumor perfusion as described above.

In each experiment, B16-F10 murine melanoma cells are injected subcutaneously into 7 groups of rats consisting of 5 rats per group. The groups are three experimental test groups of animals treated with various concentrations of an HDAC inhibitor only (single agent, groups I, II, and III), three groups of animals administered a constant amount of a B vitamin molecule in combination with various concentrations of the HDAC inhibitor (combination, groups IV, V, and VI), and a control administered vehicle only (group VII), as described in Table 3 below.

The HDAC inhibitor and the B vitamin molecule are administered sc one day prior to injection of tumor cells. High levels of HDAC inhibitor are about 100 mg/kg body weight, or at least about 50 mg/kg, 60 mg/kg, 70 mg/kg, 80 mg/kg, or about 90 mg/kg administered po or iv. Low levels are about 1 mg/kg total body weight, or less than about 2 mg/kg, less than about 3 mg/kg, or less than about 5 mg/kg. Intermediate levels are greater than about 10 mg/kg body weight, greater than about 20 mg/kg, about 30 mg/kg, or about 40 mg/kg. The HDAC inhibitor is administered adjacent to the site of administration to the single agent groups (groups I-III) over the course of 14 days at a high dose (group I), at an intermediate dosage (group II); and at a lesser dosage over the course of 14 days (group III). The groups that are administered a combination of the HDAC inhibitor and a B vitamin molecule (groups IV, V, and VI) are administered the combination from either a single injection or separately from two injections. The HDAC inhibitor for combination groups IV, V, and VI is administered at a higher dosage over the course of 14 days (Group IV); an intermediate dosage over the course of 14 days (group VI); and at a lesser dosage, over the course of 14 days (group VII). The B vitamin molecule is administered at a uniform high amount over the course of 14 days for each of combination groups IV, V, and VI. The Control group is administered vehicle only (PBS with a comparable amount of 2-hydroxypropyl-β-cyclodextrin as is used to complex with the HDAC inhibitor).

TABLE 3
Compound(s) administered to each group of animals

| | | | | Concentration |
|---|---|---|---|---|
| | | | Concentration of | of B vitamin |
| | | | HDAC inhibitor | molecule |
| Group Number | Group Name | Treatment compounds | administered | administered |
| I | Single agent | HDAC inhibitor only | high | 0 |
| | | (high level) | | |
| II | Single agent | HDAC inhibitor only | intermediate | 0 |
| | | (medium level) | | |
| III | Single agent | HDAC inhibitor only | low | 0 |
| | | (low level) | | |
| IV | Combination | Combination of HDAC | high | high |
| | | inhibitor (high level) and | | |
| | | B vitamin molecule | | |
| V | Combination | Combination of HDAC | intermediate | high |
| | | inhibitor (medium level) | | |
| | | and B vitamin molecule | | |
| VI | Combination | Combination of HDAC | low | high |
| | | inhibitor (low level) and | | |
| | | B vitamin molecule | | |
| VII | Control | PBS with a comparable | 0 | 0 |
| | | amount of 2- | | |
| | | hydroxypropyl-β- | | |
| | | cyclodextrin as used to | | |
| | | complex with the HDAC | | |
| | | inhibitor | | |

It is observed that appearance or growth of tumors in all groups that are administered the HDAC inhibitor (groups I-VI) is inhibited in comparison to the control group (group VII). Therapeutic treatment using a combination of the HDAC inhibitor and the B vitamin molecule (groups IV through VI) is found to be more efficacious than single agent treatment with the HDAC inhibitor.

Combination group IV which is administered the HDAC inhibitor at the high level and the B vitamin molecule shows the greatest amount of inhibition of tumor growth, i.e., statistically correlated with reduction in tumor size, tumor weight, tumor number, and tumor perfusion. Combination group V which is administered the HDAC inhibitor at intermediate level and the B vitamin shows greater inhibition of tumor growth, i.e., reduction in tumor size, tumor weight, tumor number, and tumor perfusion, compared to single agent group II which is administered only the HDAC inhibitor at the intermediate level and single agent group I which is administered only the HDAC inhibitor at the high level. Combination group VI which is administered the HDAC inhibitor at the low level and the B vitamin shows greater inhibition of tumor growth, i.e., reduction in tumor size, tumor weight, tumor number, and tumor perfusion, compared to single agent group III which is administered only the HDAC inhibitor at the low level, single agent group II which is administered only the HDAC inhibitor at the intermediate level, and single agent group I which is administered only the HDAC inhibitor at the high level.

These data show that there is a synergistic effect between HDAC inhibitors and B vitamin molecules in inhibiting tumors.

### Example 3

### In Vitro Analysis of Side Effects from Administration of Chemotherapic Agent

It is commonly known that anti-cancer therapeutic protocols are limited in patient acceptability by actual and perceived difficulty due to side effects. Compositions that either enhance potency so that a lower dose of an anti-cancer agent can be efficacious, or that reduce side effects so that standard or higher doses obtain greater acceptability, are needed.

Accordingly, treatment related toxicity is here analyzed using bone marrow cells, by measuring side effects resulting from the above treatments, examples of such potential side effects being myelosuppression, thrombocytopenia or anemia. Myelotoxicity is evaluated using samples of bone marrow from rats in a 14-day colony-forming unit granulocyte-macrophage assay. The decrease in appearance or extent of side effects following administration of a B vitamin provides a measure of treatment-related enhancement of the antitumor potential. General blood chemistries and concentrations of blood cells including red blood cells, white blood cells, and platelets are measured for each animal.

### Bone Marrow Samples

Seven to 10-week-old rats receive a standard laboratory diet. Animals are sacrificed by CO₂ asphyxiation, marrow is aseptically flushed from the femurs, and single-cell suspensions are prepared by gentle disruption. Cells are washed with medium and adjusted to the appropriate concentration. Medium consists of Iscove's Modified Dulbecco's Medium containing 25 mmol/L HEPES buffer and 5% (v/v) fetal bovine serum.

### Preparation of an HDAC Inhibitor and a B Vitamin Molecule

The HDAC inhibitor and the B vitamin molecule are weighed and dissolved in dimethyl sulfoxide (DMSO) from Fisher Scientific (Fair Lawn, N.J.). Serial dilutions are made in DMSO for subsequent addition to tubes containing bone marrow cells and the final DMSO concentration in all of the cultures is 0.5%.

The experiment has 5 groups, a group treated with an HDAC inhibitor only (single agent, group VIII), three groups administered the HDAC inhibitor in combination with various concentrations of a B vitamin molecule (combination, groups IX, X, and XI), and a control treated with vehicle only (group XII), as described in Table 4 below.

TABLE 4
Compound(s) administered to each group of animals

| | | | | | Concentration | |
|---|---|---|---|---|---|---|
| | | | Concentration of | | of B vitamin | |
| | | | HDAC inhibitor | | molecule | |
| Group Number | Group Name | Treatment compounds | administered | | administered | |
| VIII | Single agent | HDAC inhibitor only | 100 | pmol/liter | 0 | |
| IX | Combination | Combination of HDAC | 100 | pmol/liter | 200 | pmol/liter |
| | | inhibitor and B vitamin | | | | |
| | | molecule (low level) | | | | |
| X | Combination | Combination of HDAC | 100 | pmol/liter | 500 | pmol/liter |
| | | inhibitor and B vitamin | | | | |
| | | molecule (medium level) | | | | |
| XI | Combination | Combination of HDAC | 100 | pmol/liter | 1000 | pmol/liter |
| | | inhibitor and B vitamin | | | | |
| | | molecule (high level) | | | | |
| XII | Control | PBS with 0.5% DMSO | 0 | | 0 | |

The concentration of the HDAC inhibitor administered to single agent group VIII and in each of combination groups IX, X, and XI is 100 μmol/liter. The concentration of the B vitamin molecule administered in each of combination groups IX, X, and XI is 200 pmol/liter (group IX), 500 μmol/liter (group X), and 1000 pmol/liter (group XI). The control group of cells are administered a PBS solution containing 0.5% DMSO (group XII).

### In Vitro Granulocyte-Macrophage Assay

Bone marrow samples are collected in sterile, preservative free heparin tubes and separated by Ficoll-Hypaque (d=1070) density gradient centrifugation. The granulocyte-macrophage assay is performed as described by Iscove et al., Am J Cell Physiol 1974; 83:309-20. Briefly, 2×10⁵ bone marrow cells/ml in Iscove's modified Dulbecco's medium are plated on 35 mm Petri dishes in 0.9% methylcellulose containing 10% phytohemagglutinin stimulated leucocyte conditioned medium, 10% bovine serum albumin, and 10% human AB serum. Cultures are incubated at 37° C. in a fully humidified atmosphere with 5% CO₂. The HDAC inhibitor is included in the medium for each of groups VIII-XI for the entire culture period (14 days), and the B vitamin molecule is further included in the medium for each of groups IX-XI for the entire culture period (14 days). Granulocyte-macrophage colonies are scored on day 14 under an inverted microscope. Aggregates containing more than 40 cells are scored as colonies and aggregates containing four to 40 cells are scored as clusters.

### Results

The greatest number of colonies is found in the control group (group XII) administered only vehicle, and this number is used to normalize data obtained for the other groups, to obtain a percent survival. Therapeutic treatment using a combination of an HDAC inhibitor and a B vitamin molecule is found to produce a significantly greater number of colonies compared to the number of colonies produced from the single agent group (VIII) which is administered only the HDAC inhibitor. Combination group XI which is administered the B vitamin molecule at a concentration of 1000 μmol/liter (high level) produces the greatest number of colonies (highest percent survival) of all the groups administered the HDAC inhibitor. Even combination group IX which is administered the B vitamin molecule at a concentration of 200 pmol/liter (low level) produces a greater number of colonies than single agent group VIII administered only the HDAC inhibitor.

These data show that administration of a B vitamin in combination with an HDAC inhibitors reduces or ameliorates certain side effects associated with administration of a chemotherapeutic agent. A result of reduction or amelioration of side effects is that larger doses of the HDAC inhibitor can be used, alone, or in combination with other known anti-cancer agents, improving the therapeutic index of the HDAC inhibitor.

### EQUIVALENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. Other aspects, advantages, and modifications considered to be within the scope of the following claims.

1. A method of treating a subject having a tumor, a cell mass or a target cell, comprising administering to the subject a histone deacetylase (HDAC) inhibitor and a B vitamin molecule.

2. The method according to claim 1, wherein administering the B vitamin molecule in combination with the HDAC inhibitor decreases dose-limiting toxicity in the subject compared to the HDAC inhibitor alone, thereby allowing administration of a higher, more therapeutic dose of the HDAC inhibitor than otherwise tolerated by the subject.

3. The method according to claim 2, wherein observing decrease in proliferation of the tumor, cell mass or target cell further comprises analyzing inhibition of at least one parameter selected from the group of: tumor size; metastasis; tumor necrosis; cell proliferation rate; and cell apoptosis.

4. The method according to claim 1, wherein the subject is a mammal or mammalian cell.

5. The method according to claim 4, wherein the subject is a human.

6. The method according to claim 1, wherein the tumor, cell mass or target cell is present in at least one disease selected from the group of: a proliferative disease, a hyperproliferative disease, a cardiovascular disease, a disease of the immune system, a disease of the central nervous system, a disease of the peripheral nervous system, and a disease associated with misexpression of a gene.

7. (canceled)

8. The method according to claim 6, wherein the proliferative disease is a benign or malignant tumor, a carcinoma of the brain, kidney, liver, adrenal gland, bladder, breast, stomach (especially gastric tumors), ovaries, esophagus, colon, rectum, prostate, pancreas, lung, vagina, thyroid, sarcoma, glioblastomas, lymphoma, multiple myeloma or gastrointestinal cancer, colon carcinoma or colorectal adenoma, a tumor of the neck and head, an epidermal hyperproliferation, psoriasis, prostate hyperplasia, a neoplasia, preferably mammary carcinoma, or a leukemia.

9. The method according to claim 6, wherein the hyperproliferative disease is at least one selected from the group of: leukemias, lymphomas, hyperplasias, fibrosis (including pulmonary, and also other types of fibrosis, such as renal fibrosis), angiogenesis, psoriasis, atherosclerosis and smooth muscle proliferation in the blood vessels, such as stenosis or restenosis following angioplasty.

10. The method according to claim 6, wherein the immune condition is at least one selected from the group of: rheumatoid arthritis, Crohn's disease, multiple sclerosis, psoriasis, and Type I diabetes.

11. The method according to claim 6, wherein the immune condition is immune rejection of a transplanted allogenic graft of organ or tissue.

12. The method according to claim 6, wherein the disease to be treated is associated with persistent angiogenesis, such as psoriasis; Kaposi's sarcoma; restenosis, e.g., stent-induced restenosis; endometriosis; Crohn's disease; Hodgkin's disease; leukemia; arthritis, such as rheumatoid arthritis; hemangioma; angiofibroma; eye diseases, such as diabetic retinopathy and neovascular glaucoma; renal diseases, such as glomerulonephritis; diabetic nephropathy; malignant nephrosclerosis; thrombotic microangiopathic syndromes; transplant rejections and glomerulopathy; fibrotic diseases, such as cirrhosis of the liver; mesangial cell-proliferative diseases; arteriosclerosis; injuries of the nerve tissue; and for inhibiting the re-occlusion of vessels after balloon catheter treatment, for use in vascular prosthetics or after inserting mechanical devices for holding vessels open, such as, e.g., stents, as immunosuppressants, as an aid in scar-free wound healing, and for treating age spots and contact dermatitis.

13. The method according to claim 1, wherein the tumor, cell mass or target cell is associated with an HDAC dependent disease, wherein the HDAC is at least one selected from the group of HDAC1, HDAC2, HDAC3, HDAC4, HDAC5, HDAC6, HDAC7, HDAC8, HDAC9, HDAC10 and HDAC11.

14. The method according to claim 13, wherein the protein HDAC is at least one selected from the group of HDAC1, HDAC2, HDAC6 and HDAC8.

15.-25. (canceled)

26. The method according to claim 1 wherein the B vitamin molecule is at least one selected from the group of vitamin B1, vitamin B2, vitamin B3, vitamin B5, vitamin B6, vitamin B9 (folate), and vitamin B12.

27. (canceled)

28. The method according to claim 1 wherein the B vitamin molecule is a B vitamin precursor.

29. The method according to claim 1 wherein the B vitamin molecule is a B vitamin analog or derivative.

30. The method according to claim 1, wherein the administering is by a route that is at least one selected from the group of oral, subcutaneous, intramuscular and intravenous delivery.

31. (canceled)

32. The method according to claim 1, wherein administering the combination is administering the vitamin and the inhibitor simultaneously.

33. The method according to claim 1, wherein administering the combination is administering the vitamin and the inhibitor sequentially.

34. The method according to claim 1, wherein the treatment is administering doses of the vitamin and the inhibitor at different frequencies.

35.-36. (canceled)

37. The method according to claim 1, wherein the dose of the vitamin is selected from: at least 50 μg/subject/dose, at least 500 μg/subject/dose, at least 50 mg/subject/dose, at least 50 mg/subject/dose, and at least 500 mg/subject/dose.

38.-41. (canceled)

42. The method according to claim 1, wherein administering further comprises an amount of the HDAC inhibitor/subject/day that is greater and produces fewer side effects than the same amount absent the vitamin.

43. A use of a combination of an HDAC inhibitor and a B vitamin molecule as an anti-cancer treatment, wherein the B vitamin molecule in combination with the HDAC inhibitor alleviates dose-limiting toxicity, thereby resulting in tolerance by the subject of a higher, more therapeutic dose of the HDAC inhibitor compared to administration of the HDAC inhibitor alone.

44. The use according to claim 43, further comprising measuring inhibition of at least one parameter selected from the group consisting of: rate of increase in tumor size; rate of increase in tumor number (metastasis); and rate of proliferation of transformed cells.

45. A kit comprising each of an HDAC inhibitor, a B vitamin molecule, and a container.

46.-49. (canceled)

50. A composition comprising an HDAC inhibitor and a B vitamin molecule.

51. The composition according to claim 50 wherein each of the HDAC inhibitor and the B vitamin molecule is present in an effective dose.

52. The composition according to claim 50 further comprising a pharmaceutically acceptable buffer.

53. The composition according to claim 50 wherein the composition is in a unit dose.

54. The composition according to claim 50, further comprising an additional agent which is an anti-proliferative agent.

55. The composition according to claim 50, wherein
a) the HDAC inhibitor is selected from the group of compounds including hydroxamic acid, hydroxamate, hydroxyamide, cyclic peptide, anti-HDAc antibody, benzamide, benzimidazole, short-chain fatty acid, mercaptomide, carbamic acid, carbonyl, piperazinyl, piperidinyl, morpholinyl, sulfonyl, amine, amide, valproic acid, oxime, dioxane, epoxide, lactam, and depudecin, especially N-(2-aminophenyl)-4-[N-(pyridin-3-ylmethoxycarbonlyl)aminomethyl]benzamide, N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(2-methyl-1H-indol-3-yl)-ethyl]-amino]methyl]phenyl]-2E-2-propenamide and pharmaceutically acceptable salts thereof; and
b) the B vitamin molecule is selected from the group of vitamin B2, vitamin B3, vitamin B6, vitamin B9 (folate), and vitamin B12.

56.-62. (canceled)
